(19) United States
(12) Patent Application Publication
(10) Pub. No.: US 2024/0210399 A1
(43) Pub. Date: Jun. 27, 2024

(54) METHODS FOR ISOLATING AND ANALYZING A TARGET ANALYTE ENCAPSULATED BY A SURFACE MARKER DISPLAYING AGENT

(71) Applicant: MESO SCALE TECHNOLOGIES, LLC., Rockville, MD (US)

(72) Inventors: Pankaj OBEROI, Rockville, MD (US); David ROUTENBERG, Gaithersburg, MD (US); Alexander TUCKER-SCHWARTZ, Bethesda, MD (US)

(21) Appl. No.: 18/557,216

(22) PCT Filed: Apr. 20, 2022
(86) PCT No.: PCT/US2022/025505

§ 371 Date: Oct. 25, 2023

Related U.S. Application Data
(60) Provisional application No. 63/179,730, filed on Apr. 26, 2021.

(51) Int. Cl. G01N 33/569; G01N 33/53; G01N 33/543

(52) U.S. Cl. CPC G01N 33/56983; G01N 33/5308; G01N 33/54306; G01N 2333/165; G01N 2469/10

(57) ABSTRACT

Method and kits for highly specific detection and isolation of a target analyte encapsulated by a surface marker displaying agent (SMDA) are disclosed.

### FIELD OF THE INVENTION

The invention relates to methods and kits for isolating and analyzing a target analyte of interest encapsulated by a surface marker displaying agent (SMDA).

### BACKGROUND

Surface marker displaying agents (SMDA) include cells, viruses and viral particles, cellular organelles, and vesicles, including extracellular vesicles (EVs) and exosomes. SMDA may include biologically relevant materials or components. Methods of isolating or characterizing various types of SMDA are actively being developed.

Extracellular vesicles (EVs) are a diverse group of cell-secreted membrane vesicles implicated in a wide variety of physiological and pathological processes, many of which are only beginning to be understood. Although initially thought to function in the removal of unwanted molecules from cells, EVs are now recognized as important mediators of cell-cell communication and are involved in processes such as immune regulation, antigen presentation, tumor progression and metastasis, modulation of inflammation, stem cell regulation, neuronal development and regeneration, and cell-to-cell transfer of pathogenic proteins and nucleic acids. EVs are secreted from nearly all cell types through multiple mechanisms including the fusion of specific endosomal compartments called multivesicular bodies (MVB) with the plasma membrane and by budding/shedding directly from the plasma membrane. EVs are present in nearly all body fluids including blood, urine, cerebral spinal fluid, and saliva, and are secreted by most in vitro cultured cells as well. Because of the EV formation mechanisms, EVs contain specific lipids, membrane proteins, and internalized proteins, nucleic acids and metabolites derived from their cells of origin and are thus a rich source of potential biomarkers.

Recent research suggests a role for EVs in the function of the healthy central nervous system (CNS) as well as a role in numerous diseases of the CNS. Many cells of the CNS including neurons, astrocytes, oligodendroglia and microglia have been shown to secrete EVs in vitro. In neurons, synaptic activity-dependent EV release and reuptake has been observed and has been proposed as a possible mechanism of synaptic plasticity and inter-neuronal transfer of complex information. Neurons have also been shown to transfer miRNA via EVs to astrocytes, modulating the level of an important functional synaptic protein, EAAT2/GLT1. EV secretion by oligodendrocytes has been found to modulate myelin biogenesis, promote neuronal viability under stress and enable degradation of oligodendroglial membrane proteins by a subset of microglia through an “immunologically silent” macropinocytotic mechanism. Astrocyte-derived EVs have been shown to promote neuronal survival under stress by transferring heat-shock proteins and synapsin I. EV secretion by microglia has been shown to be inducible by Wnt-signaling and to stimulate synaptic activity by enhancing sphingosine metabolism in neurons and to represent a unique secretion mechanism for IL-1beta, an important neuroinflammatory cytokine.

In addition to promoting healthy CNS function, EVs appear to play several roles in various CNS diseases and disorders. Broadly these include the export of toxic proteins and possibly promotion of toxic isoform formation, mediation of neuroinflammation, and the transfer of disease associated miRNAs. Numerous studies have demonstrated that EVs can mediate the transfer of toxic proteins between cells both in-vitro and in animal studies. This includes the misfolded prion protein PrPSc, the infectious agent in human diseases Creutzfeldt-Jakob disease (CJD) and Gerstmann-Sträussler-Scheinker syndrome (GSS), aggregated alpha-synuclein, the pathogenic species associated with Parkinson's disease and Lewy body dementia, aggregated Tau and beta-amyloid peptides hallmarks of Alzheimers disease (AD), frontotemporal lobar degeneration (FTD) and progressive supranuclear palsy (PSP), and mutated SOD1, linked to the development of amyotrophic lateral sclerosis (ALS). There is also some evidence that the secretion of toxic proteins in EVs may actually have a protective role, facilitating clearing of these pathogenic species by microglia.

EVs are known to contain cell-type specific cargo, including, for example, RNA, DNA and proteins. EVs can interact with cells, for example, through ligand-receptor interaction, release of vesicle contents in the extracellular space, direct fusion with the plasma membrane and endocytosis into the cell. Mechanisms that result in a transfer of molecular cargo from EVs to the recipient cells are of particular interest. Jabalee et al. (2018) The role of extracellular vesicles in cancer: cargo, function, and therapeutic implications. Cells. 7(8):93 (doi: 10.3390/cells7080093)

Assessing the composition of SMDAs and their encapsulated cargo requires appropriate isolation and purification methods. Currently, the most popular method of EV isolation remains differential ultracentrifugation (DU). Although DU is a low-cost, high-throughput method, co-purification of non-vesicular proteins and other contaminants is an issue. Jabalee et al. (2018) The role of extracellular vesicles in cancer: cargo, function, and therapeutic implications. Cells. 7(8):93 (doi: 10.3390/cells7080093). While combination of DU with other purification methods, such as ultrafiltration or density gradient centrifugation can improve the purity of the collected vesicles, particle yield is diminished.

Once EVs have been isolated and purified, the encapsulated cargo must be extracted and profiled. However, measuring one or more target analytes encapsulated by SMDAs can be difficult for several reasons. First, many of the proteins are also present outside the SMDA in a soluble or aggregated form, sometimes in much higher concentration than in the SMDA lumen. Therefore, a method to separate SMDA-associated protein from soluble protein is required. Second, the SMDA must be lysed to access the cargo proteins using lysis conditions that are compatible with the assay. Moreover, methods that are scalable and amenable to automation are desirable.

### SUMMARY OF THE INVENTION

In one aspect, a method of isolating, detecting or quantifying one or more target analytes of interest encapsulated by one or more surface marker displaying agents (SMDA) in a sample is provided. In one aspect, the method includes contacting a support surface with the sample that contains the SMDA encapsulating the target analyte, wherein the support surface includes a plurality of binding domains and the SMDA selectively binds to a SMDA binding domain on the support surface. In one aspect, the SMDA selectively binds to a SMDA capture reagent immobilized on a SMDA binding domain of the support surface, wherein the SMDA is immobilized on the SMDA binding domain.

In one aspect, the method includes removing unwanted components of the sample from the support surface after the SMDA is immobilized on the support surface. In one aspect, the unwanted components include soluble analyte present in the sample that is not encapsulated by the SMDA. In one aspect, the method includes a wash step for removing unwanted components of the sample from the support surface.

In one aspect, the support surface is contacted with a lysis reagent to lyse the immobilized SMDA and release the encapsulated target analyte. In one aspect, the target analyte selectively binds to a target analyte binding domain on the support surface. In one aspect, the target analyte selectively binds to a target analyte capture reagent to form a target analyte binding complex that includes the target analyte and the target analyte capture reagent. In one aspect, the target analyte binding complex is formed in solution. In one aspect, the target analyte capture reagent is immobilized on a target analyte binding domain of the support surface and the target analyte binding complex is formed on the support surface. In one aspect, the target analyte capture reagent is directly immobilized on a target analyte binding domain of the support surface. In one aspect, the target analyte capture reagent is indirectly immobilized on a target analyte binding domain.

In one aspect, the target analyte selectively binds to a target analyte capture reagent that includes a targeting reagent that binds to a targeting reagent complement immobilized on a target analyte binding domain of the support surface and is thereby immobilized on the support surface. In one aspect, the target analyte capture reagent is immobilized on the support surface and binds to the target analyte after the sample has been added to the support surface. In one aspect, the target analyte capture reagent is immobilized on the support surface and binds to the target analyte after unwanted components have been removed from the support surface. In one aspect, the target analyte capture reagent is added to the support surface and binds to the target analyte after the sample has been added to, and after unwanted components have been removed from, the support surface. In one aspect, the target analyte capture reagent includes a targeting reagent that selectively binds to a targeting reagent complement immobilized on the target analyte binding domain and is thereby indirectly immobilized on the target analyte binding domain of the support surface. In one aspect, the target analyte capture reagent includes a targeting reagent that selectively binds to a targeting reagent complement immobilized on the target analyte binding domain and is thereby indirectly immobilized on the target analyte binding domain of the support surface after the sample has been added to the support surface and after unwanted components have been removed from the support surface.

In one aspect, one or more of the plurality of binding domains on a support surface include a SMDA capture reagent that selectively binds SMDA after which unwanted components are removed from the support surface, the SMDA is lysed, and one or more binding domains on the support surface include a target analyte capture reagent that selectively binds target analyte released from the lysed SMDA.

In one aspect, the sample includes both target analyte that is encapsulated in the SMDA and nonencapsulated target analyte. In one aspect, the nonencapsulated target analyte includes soluble target analyte present in the sample. In one aspect, nonencapsulated target analyte that is not encapsulated by the SMDAs is an unwanted component in the sample. In one aspect, the method includes contacting the sample with a blocking reagent to remove, destroy or block an unwanted component in the sample. In one aspect, the method includes contacting the sample with a blocking reagent to remove, destroy or block an unwanted component in the sample before the wash step. In one aspect, the method includes contacting the sample with a blocking reagent to remove, destroy or block nonencapsulated target analyte in the sample. In one aspect, the blocking reagent binds to and prevents the nonencapsulated target analyte from binding to a target analyte capture reagent immobilized in a target analyte binding domain on the support surface. In one aspect, the blocking reagent includes, but is not limited to, an antibody, antigen-binding antibody fragment or other antigen-binding reagent that specifically binds to the nonencapsulated target analyte. In one aspect, the blocking reagent includes, but is not limited to, an oligonucleotide or aptamer that specifically binds to the nonencapsulated target analyte. In one aspect, the blocking reagent includes an enzyme, including, but not limited to, a protease, nuclease or glycosidase that degrades the nonencapsulated target analyte.

In one aspect, the SMDA capture reagent selectively binds to a surface marker of the SMDA. In one aspect, the SMDA capture reagent is immobilized on the SMDA binding domain of the support surface. In one aspect, the SMDA capture reagent is an antigen-binding substance, antibody, antigen, ligand, receptor, oligonucleotide, hapten, epitope, mimotope, or aptamer. In one aspect, the SMDA capture reagent includes an antigen-binding substance that selectively binds a surface marker on the SMDA. In one aspect, the SMDA capture reagent includes an antigen-binding substance that selectively binds a disease-specific surface marker on the SMDA. In one aspect, the SMDA capture reagent is releasably immobilized on the SMDA binding domain. In one aspect, the SMDA capture reagent is directly immobilized on the SMDA binding domain. In one aspect, the SMDA capture reagent is indirectly immobilized on the SMDA binding domain. In one aspect, the SMDA capture reagent includes a targeting reagent that selectively binds to a targeting reagent complement immobilized on the SMDA binding domain of the support surface, thereby indirectly immobilizing the SMDA capture reagent on the support surface. In one aspect, the targeting reagent and the targeting reagent complement include complementary oligonucleotide sequences.

In one aspect, the SMDA capture reagent is releasably bound to the SMDA binding domain by a labile linker. In one aspect, the labile linker is a heat-labile linker, a photolabile linker, or a chemically labile linker. In one aspect, the labile linker includes an oligonucleotide that includes a restriction site cleavable by a restriction endonuclease.

In one aspect, the method includes releasing the SMDA capture reagent from the support surface after the support surface is contacted with the lysis reagent. In one aspect, the method includes releasing the immobilized SMDA from the support surface after the support surface is contacted with the lysis reagent.

In one aspect, the method includes contacting the immobilized SMDA with a SMDA detection reagent that specifically binds to the immobilized SMDA to detect or quantify the SMDA. In one aspect, the method includes contacting the immobilized SMDA with a SMDA detection reagent to detect or quantify the SMDA before the support surface is contacted with the lysis reagent. In one aspect, the method includes contacting the immobilized SMDA with a SMDA detection reagent to detect or quantify the SMDA after the support surface is contacted with the lysis reagent. In one aspect, the SMDA detection reagent specifically binds to a surface marker of the SMDA. In one aspect, the SMDA detection reagent includes a label. In one aspect, the SMDA detection reagent binds to a surface marker of the SMDA that is bound to a SMDA capture reagent immobilized (either directly or indirectly) on a support surface. In one aspect, the SMDA detection reagent and surface marker remain bound to the immobilized SMDA capture reagent after the support surface is contacted with a lysis reagent. In one aspect, the SMDA detection reagent and surface marker remain bound to the immobilized SMDA capture reagent after the lysis reagent lyses the SMDA. In one aspect, the SMDA detection reagent includes an antigen-binding substance, antigen, ligand, receptor, oligonucleotide, hapten, epitope, mimotope, or aptamer. In one aspect, the SMDA detection reagent includes an antigen-binding substance that selectively binds a surface marker on the SMDA. In one aspect, the SMDA detection reagent includes an antigen-binding substance that selectively binds a cell-specific surface marker on the SMDA. In one aspect, the SMDA detection reagent includes an antigen-binding substance that selectively binds a disease-specific surface marker on the SMDA. In one aspect, the SMDA detection reagent includes an antibody or an antigen-binding antibody fragment. In one aspect, the SMDA detection reagent includes a labeled antibody or an antigen-binding antibody fragment. In one aspect, the SMDA detection reagent includes a labeled antibody or an antigen-binding antibody fragment that specifically binds to a surface marker of the SMDA.

In one aspect, the method includes contacting the support surface with a target analyte capture reagent that selectively binds to the target analyte. In one aspect, the target analyte capture reagent includes a targeting reagent that is capable of selectively binding to a targeting reagent complement immobilized on a target analyte binding domain of the support surface. In one aspect, the target analyte capture reagent is an antigen-binding substance, antibody, antigen, ligand, receptor, oligonucleotide, hapten, epitope, mimotope, or aptamer. In one aspect, the target analyte capture reagent includes an antigen-binding substance that selectively binds the target analyte. In one aspect, the target analyte capture reagent includes a targeting reagent that selectively binds to a targeting reagent complement, wherein the targeting reagent complement is a binding partner of the targeting reagent. In one aspect, the targeting reagent of the target analyte capture reagent and the targeting reagent complement immobilized on the target analyte binding domain include complementary oligonucleotide sequences.

In one aspect, one or more binding domains on the support surface include different targeting reagent complements. In one aspect, one or more binding domains on the support surface include different SMDA capture reagents. In one aspect, one or more binding domains on the support surface include different target analyte capture reagents. In one aspect, one or more different target analyte capture reagents are directly immobilized in one or more binding domains. In one aspect, one or more different target analyte capture reagents are indirectly immobilized in one or more binding domains, for example, through the binding of a targeting reagent appended to the target analyte capture reagent and a targeting reagent complement immobilized on the one or more target analyte binding domains. In one aspect, the support surface includes more than one target analyte binding domain and each target analyte binding domain includes a different immobilized target analyte capture reagent than the other target analyte binding domains. In one aspect, the support surface includes more than one target analyte binding domain and each target analyte binding domain includes a different immobilized targeting reagent complement than the other target analyte binding domains.

In one aspect, unwanted components of the sample do not bind to the support surface, the SMDA capture reagent or the target analyte capture reagent. In one aspect, unwanted components including unbound, soluble analytes are removed from the support surface before contacting the support surface with the target analyte capture reagent. Unwanted components including unbound, soluble analytes may be removed, for example, by washing the support surface under conditions wherein SMDAs that have been immobilized to the support surface remain immobilized.

In one aspect, the method includes removing unwanted components of the SMDA from the support surface after the SMDA is lysed. In one aspect, the unwanted components of the SMDA do not bind to the support surface, the SMDA capture reagent or the target analyte capture reagent.

In one aspect, the method includes conducting an assay to identify, detect, or quantify, one or more SMDA or one or more target analytes. In one aspect, the method includes conducting an assay to identify, detect, or quantify, one or more cell surface markers or one or more target analytes encapsulated by the SMDA.

In one aspect, the SMDA encapsulates more than one target analyte of interest. In one aspect, the method includes assaying more than one target analyte. In one aspect, the support surface includes a different target analyte binding domain with a different target analyte capture reagent or a different targeting reagent complement for each target analyte of interest. In one aspect, each target analyte selectively binds, either directly or indirectly, to their corresponding binding domain on the support surface. In one aspect, the method includes contacting the support surface with a plurality of different target analyte capture reagents, wherein each target analyte capture reagent selectively binds a different target analyte of interest. In one aspect, the target analyte capture reagent binds to the target analyte in solution to form a target analyte binding complex. In one aspect, the target analyte binding complex is formed in solution and then immobilized in a binding domain on the support surface. In one aspect, the capture reagent includes a targeting reagent that is capable of selectively binding to a corresponding targeting reagent complement immobilized on a target analyte binding domain on the support surface. In one aspect, the target analyte capture reagent is directly immobilized in a target analyte binding domain on a support surface and the target analyte binding complex is formed on the support surface.

In one aspect, the SMDA is a cell, a virus or viral particle, an organelle, a vesicle, or combination thereof. In one aspect, the SMDA is a cell. In one aspect, the cell is a bacterial cell. In one aspect, the cell is a eukaryotic cell. In one aspect, the eukaryotic cell is a human cell. In one aspect, the SMDA is a viral particle. In one aspect, the SMDA is a vesicle. In one aspect, the vesicle includes an extracellular vesicle (EV). In one aspect, the EV is an exosome, a micro-vesicle, an ectosome, an apoptotic body, or a large-oncosome.

In one aspect, the target analyte is a protein, a peptide, a nucleic acid, a carbohydrate, a lipid, a hormone, a metabolite, or a combination thereof. In one aspect, the target analyte includes a disease specific marker. In one aspect, the target analyte includes a protein. In one aspect, the target analyte includes a nucleic acid. In one aspect, the nucleic acid is DNA or RNA. In one aspect, the nucleic acid includes mRNA. In one aspect, the nucleic acid includes miRNA.

In one aspect, the sample includes purified SMDA. In one aspect, the sample includes a mammalian fluid, secretion, or excretion. In one aspect, the sample includes a purified mammalian fluid, secretion, or excretion. In one aspect, the mammalian fluid, secretion, or excretion is purified by precipitation, differential centrifugation, ultrafiltration, size-exclusion chromatography, immune-affinity, or combination thereof. In one aspect, the mammalian fluid, secretion, or excretion is, for example, whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions, excretions, and the like.

In one aspect, the method includes contacting the target analyte bound to the target analyte capture reagent with a target analyte detection reagent that specifically binds to the target analyte to detect or quantify the target analyte. In one aspect, the target analyte detection reagent includes a nucleic acid probe. In one aspect, the method includes forming a target analyte detection complex that includes the target analyte capture reagent, the target analyte, and the target analyte detection reagent. In one aspect, the method includes extending the nucleic acid probe of the target analyte detection reagent to form an extended sequence. In one aspect, the method includes detecting or measuring the amount of extended sequence.

In one aspect, extending the nucleic acid probe includes contacting the nucleic acid probe with a circular template oligonucleotide and forming the extended sequence by rolling circle amplification. In one aspect, the extended sequence includes a detection sequence complement that is complementary to a sequence of a detection oligonucleotide. In one aspect, the method includes measuring the amount of extended sequence immobilized on the support surface by contacting the extended sequence with a detection oligonucleotide and measuring the amount of detection oligonucleotide immobilized on the target analyte binding domain. In one aspect, the detectable label includes an electrochemiluminescent label (ECL). In one aspect, the detectable label includes a fluorescent label.

In one aspect, the support surface is a single component with a plurality of binding domains. In one aspect, the support surface includes a plate that has multiple wells, and each well includes at least one binding domain. In one aspect, each well includes multiple SMDA binding domains that include SMDA capture reagent immobilized thereon. In one aspect, each well includes multiple target analyte binding domains that include target analyte capture reagent immobilized thereon. In one aspect, each well includes multiple SMDA binding domains that include targeting reagent complement immobilized thereon. In one aspect, each well includes multiple target analyte binding domains that include targeting reagent complement immobilized thereon. In one aspect, each well includes multiple binding domains, at least one of which is a SMDA binding domain that includes SMDA capture reagent immobilized thereon and at least one of which are target analyte binding domains that include target analyte capture reagent immobilized thereon. In one aspect, each well includes multiple binding domains, at least one of which is a SMDA binding domain that includes SMDA capture reagent directly or indirectly immobilized thereon and at least one of which are target analyte binding domains that include targeting reagent complement immobilized thereon. In one aspect, at least one SMDA binding domain and at least one target analyte binding domain are in the same well. In one aspect, the SMDA binding domains and the target analyte binding domain are in different wells.

In one aspect, the support surface includes multiple components, and each of the multiple components includes at least one binding domain. In one aspect, the support surface includes multiple particles or beads, and each binding domain of the plurality of binding domains is on a separate particle or bead.

In one aspect, the support surface includes an electrode.

In one aspect, a kit is provided for isolating, detecting, quantifying, or a combination thereof, one or more SMDA and one or more target analytes of interest encapsulated by one or more SMDA in a sample. In one aspect, the kit includes a support surface. In one aspect, the kit includes a support surface with a plurality of binding domains. In one aspect, the kit includes, in one or more vials, containers, or compartments a SMDA capture reagent that binds, directly or indirectly, a surface marker of the SMDA. In one aspect, the SMDA capture reagent is immobilized on a SMDA binding domain on the support surface. In one aspect, the kit includes, in one or more vials, containers, or compartments, a target analyte capture reagent that binds, directly or indirectly, the target analyte. In one aspect, the target analyte capture reagent is immobilized on a target analyte binding domain on the support surface. In one aspect, the target analyte capture reagent includes a targeting reagent that specifically binds to a targeting reagent complement that is immobilized on a target analyte binding domain on the support surface.

In one aspect, the kit includes a SMDA capture reagent that selectively binds to a surface marker of the SMDA. In one aspect, the SMDA capture reagent includes an antigen-binding substance, antigen, ligand, receptor, oligonucleotide, hapten, epitope, mimotope, or aptamer. In one aspect, the SMDA capture reagent includes an antigen-binding substance. In one aspect, the SMDA capture reagent is immobilized on the support surface. In one aspect, the SMDA capture reagent includes a targeting reagent capable of binding to a targeting reagent complement immobilized on the SMDA binding domain. In one aspect, the targeting reagent and the targeting reagent complement include complementary oligonucleotides.

In one aspect, the kit includes a target analyte capture reagent that selectively binds the target analyte. In one aspect, the target analyte capture reagent includes an antigen-binding substance, antigen, ligand, receptor, oligonucleotide, hapten, epitope, mimotope, or aptamer. In one aspect, the target analyte capture reagent includes an antigen-binding substance. In one aspect, target analyte capture reagent is immobilized on the support surface. In one aspect, the target analyte capture reagent includes a targeting reagent capable of binding to a targeting reagent complement immobilized on the target analyte binding domain. In one aspect, the targeting reagent and its corresponding targeting reagent complement include complementary oligonucleotides.

In one aspect, one or more binding domains of the support surface include different capture reagents. In one aspect, one or more binding domains of the support surface include different targeting reagent complements.

In one aspect, the SMDA capture reagent is releasably bound to the support surface.

In one aspect, the support surface includes a single component including a plurality of binding domains. In one aspect, the support surface includes a plate including multiple wells, and each well includes at least one binding domain. In one aspect, the support surface includes a plurality of beads, and each binding domain of the plurality of binding domains is on a separate bead. In one aspect, the support surface includes an electrode.

### BRIEF DESCRIPTION OF THE DRAWINGS

The following drawings form part of the present specification and are included to further demonstrate certain aspects of the methods and kits described herein.

FIGS. 1A-1D provide a schematic illustration of a method described herein. FIG. 1A: shows SMDA captured on a support surface by a SMDA capture reagent. FIG. 1B shows SMDA captured on a support surface after unwanted components of the sample are removed with a wash. FIG. 1C: illustrates the support surface being contacted with a lysis reagent to lyse the SMDA and release the target analyte (Δ). In the method shown in FIG. 1C, the support surface is contacted with a target analyte capture reagent that selectively binds to the target analyte in solution to form a target analyte binding complex. FIG. 1D: shows the target analyte captured on the support surface by direct immobilization of the capture reagent onto the support surface, wherein the target analyte capture reagent, target analyte and target analyte detection reagent form a target analyte detection complex that can be detected or quantified based on the presence of a detectable label (⋆).

FIGS. 2A-2C provide a schematic illustration of direct and indirect binding of a target analyte capture reagent to a support surface. FIG. 2A: is a schematic illustration of a target analyte capture reagent that is directly immobilized onto a support surface. FIG. 2B: is a schematic illustration of a target analyte capture reagent that includes a targeting reagent (TR) that binds to a targeting reagent complement (TRC) immobilized on a support surface, indirectly immobilizing the target analyte capture reagent onto the support surface. FIG. 2C: shows the target analyte captured on the support surface by indirect immobilization of the capture reagent onto the support surface, wherein the target analyte capture reagent, target analyte and target analyte detection reagent form a target analyte detection complex that can be detected or quantified based on the presence of a detectable label (⋆).

### DETAILED DESCRIPTION

### I. Overview

Described herein are methods of isolating, detecting or quantifying a target analyte that is encapsulated by a surface marker displaying agent (SMDA). SMDAs can be naturally-occurring, partially synthetic, or fully synthetic. In one aspect, a SMDA is a biologically relevant material or component. In general, a SMDA includes a surface, such as a lipid bilayer, membrane, cell wall, or envelope, on which one or more surface markers are displayed. In one aspect, the SMDA encapsulates one or more analytes of interest, including, but not limited to, proteins, nucleic acids, including DNA or RNA, lipids, carbohydrates, small molecules such as hormones, cofactors, vitamins, minerals, salts, metals, metal-containing compounds, or combinations thereof. Examples of SMDAs include cells (including prokaryotic cells such as bacterial cells or archaeal cells; eukaryotic cells such as mammalian cells, insect cells, or plant cells); viruses and viral particles; cellular organelles such as nucleus, endoplasmic reticulum, Golgi apparatus, mitochondria, vacuoles, or chloroplast; vesicles such as lysosome, endosome, peroxisome, and liposome; and extracellular vesicles (EVs) or exosomes.

Although a variety of analytical methods have been used to characterize SMDAs and their encapsulated contents, including, for example, immunoassays (e.g., Western blotting, flow cytometry, and sandwich immunoassays), electron microscopy, mass spectrometry, PCR and sequencing, and nanoparticle tracking, there remains a need for an efficient way to detect or isolate a target analyte that is encapsulated within the SMDA.

The methods and kits provided herein can be used to isolate, detect and quantify one or more analytes of interest encapsulated in one or more SMDA(s). This can be important for understanding intercellular trafficking of pathogenic proteins and in identifying highly specific biomarkers of pathogenesis in diseases. However, most existing isolation techniques are difficult to scale for use with large numbers of samples, particularly those that require centrifugation or chromatography. In contrast, methods provided herein are scalable and amenable to automation.

Described herein is a surprisingly effective and highly specific method of isolating, detecting or quantifying a target analyte encapsulated in a SMDA. In one aspect, the method includes contacting a support surface with the sample that contains the SMDA encapsulating the target analyte. In one aspect, the support surface includes a plurality of binding domains and the SMDA selectively binds, either directly or indirectly to a SMDA binding domain on the support surface. In one aspect, the method includes removing unwanted components of the sample from the support surface. In one aspect, the support surface is contacted with a lysis reagent to lyse the SMDA and release the encapsulated target analyte. In one aspect, a target analyte capture reagent is added to the support surface to form a target analyte binding complex and the target analyte binding complex is allowed to selectively bind, either directly or indirectly, to a target analyte binding domain on the support surface. In one aspect, a target analyte capture reagent is immobilized in a target analyte binding domain on the support surface that was previously coated with immobilized SMDA capture reagent and then washed, and the target analyte binding complex is formed on the support surface.

### II. Definitions

The following definitions are intended to aid the reader in understanding the present invention but are not intended to vary or otherwise limit the meaning of such terms unless specifically indicated.

As used herein and in the appended claims, the singular forms “a,” “an,” and “the” include plural referents unless the context clearly dictates otherwise. Thus, for example, reference to “a target analyte” can refer to one or more target analytes, and reference to “the method” includes reference to equivalent steps and methods known to those skilled in the art.

As used herein, the term “or” can mean “and/or”, unless explicitly indicated to refer only to alternatives or the alternatives are mutually exclusive. The terms “including,” “includes” and “included” are not limiting.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, polymerase chain reaction, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach and Veksler, Eds. (2007), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3.sup.rd Ed., W. H. Freeman Pub., New York, N.Y.; and Berg et al. (2002) Biochemistry, 5.sup.th Ed., W.H. Freeman Pub., New York, N.Y., all of which are herein incorporated in their entirety by reference for all purposes.

“Isolating” a surface marker displaying agent (SMDA) or a target analyte encapsulated in a SMDA means separating the SMDA or the target analyte from other unwanted components of a sample. With respect to a target analyte encapsulated in a SMDA, “isolating” can refer to separating the target analyte from other unwanted components of the SMDA. In one aspect, isolating a SMDA means separating the SMDA from unwanted components of a sample that can include target analyte that is not encapsulated by the SMDA. The term “isolating” is synonymous with enriching and purifying. In one aspect, the concentration of the SMDA or target analyte in the resulting “isolate” is at least about 10×, about 25×, about 50×, about 100×, or about 200× greater than the concentration of the SMDA or target analyte in the original preparation. In one aspect, at least about 50%, about 55%, about 60%, about 65%, about 70%, about 75%, about 80%, about 85%, about 90%, about 95%, and up to about 99% of at least one unwanted component present in the original preparation is removed from the isolate. In one aspect, the “isolated” SMDA or target analyte includes undetectable levels of unwanted components when measured using known methods for detecting such components.

“Complementary” refers to nucleic acid molecules or a sequence of nucleic acid molecules that interact by the formation of hydrogen bonds, for example, according to the Watson-Crick base-pairing model. For example, “complementary” can refer to two oligonucleotides whose bases form complementary base pairs, base by base, for example, in which A pair with T or U and C pairs with G. Hybridization can occur between two complementary DNA molecules (DNA-DNA hybridization), two complementary RNA molecules (RNA-RNA hybridization), or between complementary DNA and RNA molecules (DNA-RNA hybridization). Complementary sequences need not hybridize along their entire length. For example, hybridization can occur between a short nucleotide sequence that is complementary to a portion of a longer nucleotide sequence. Hybridization can occur between sequences that do not have 100% “sequence complementarity” (i.e., sequences where less than 100% of the nucleotides align based on a base-pairing model such as the Watson-Crick base-pairing model), although sequences having less sequence complementarity are less stable and less likely hybridize than sequences having greater sequence complementarity. In one aspect, the nucleotides of the complementary sequences have 100% sequence complementarity based on the Watson-Crick model. In another aspect, the nucleotides of the complementary sequences have at least about 90%, about 95%, about 96%, about 97%, about 98% or about 99% sequence complementarity based on the Watson-Crick model.

Whether or not two complementary sequences hybridize can depend on the stringency of the hybridization conditions, which can vary depending on conditions such as temperature, solvent, ionic strength and other parameters. The stringency of the hybridization conditions can be selected to provide selective formation or maintenance of a desired hybridization product of two complementary nucleic acid sequences in the presence of other potentially cross-reacting or interfering sequences. Stringent conditions are sequence-dependent—typically longer complementary sequences selectively hybridize at higher temperatures than shorter complementary sequences. Generally, stringent hybridization conditions are between about 5° C. to about 10° C. lower than the thermal melting point (Tm) (i.e., the temperature at which 50% of the sequences hybridize to a substantially complementary sequence) for a specific nucleotides sequence at a defined ionic strength, concentration of chemical denaturants, pH and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations of less than about 1 M, about 500 mM, or about 200 mM; hybridization temperatures above about 20° C., about 30° C., about 40° C., about 60° C. or about 80ºC; and chemical denaturant concentrations above about 10%, about 20%, about 30% about 40% or about 50%. Because many factors can affect the stringency of hybridization, the combination of parameters may be more significant than the absolute value of any parameter alone.

“Detectable label” refers to a chemical group or moiety that has a detectable physical property; is capable of causing a chemical group or moiety to exhibit a detectable physical property, for example, an enzyme that catalyzes conversion of a substrate into a detectable product; or is a compound that is a member of a binding pair, in which a first member of the binding pair is attached to a structure, for example, a detection reagent, and the other member of the binding pair has a detectable physical property or is attached to a chemical group or moiety that has a detectable physical property or is capable of causing a chemical group or moiety to exhibit a detectable physical property. Non-limiting examples of binding pairs include biotin and streptavidin, or avidin; complementary oligonucleotides; hapten and hapten binding partner; and antibody-antigen binding pairs. In one aspect, a first member of the binding pair is attached to a SMDA detection reagent and the other member of the binding pair is attached to a detectable label. In one aspect, a first member of the binding pair is attached to a target analyte detection reagent and the other member of the binding pair is attached to a detectable label. In one aspect, a first member of a binding pair, for example, biotin, is attached to a SMDA detection reagent and the streptavidin or avidin is attached to a detectable label. In one aspect, a first member of a binding pair, for example, biotin, is attached to a target analyte detection reagent and the streptavidin or avidin is attached to a detectable label. In one aspect, the binding pair is a pair of complementary oligonucleotides. In one aspect, the detectable label is an electrochemiluminescent (ECL) label.

“Detection” refers to detecting, observing, or quantifying the presence of a substance, such as a SMDA or a target analyte, based on the presence or absence of a detectable label. A detectable label can be detected by spectroscopic, photochemical, biochemical, immunochemical, electrical, optical, chemical, or other methods. Examples of detectable labels include, but are not limited to, radioisotopes, enzymes, substrates, fluorescent molecules, chemiluminescent moieties, electrochemiluminescent moieties, magnetic particles, and bioluminescent moieties.

As used herein, “encapsulated” refers to a target analyte that is physically associated with or entrapped within a SMDA. In one aspect, the target analyte is located entirely within the SMDA. In one aspect, the target analyte is not exposed on the surface of the SMDA.

As used herein, “nonencapsulated” refers to a component in a sample that is not physically associated with a SMDA, for example, a component that is not a membrane-bound structure of the SMDA or entrapped within a SMDA. In one aspect, the nonencapsulated component in a sample includes target analyte that is not encapsulated within the SMDA.

“Selectively binds” or “specifically binds” means that one member of a binding pair preferentially binds to its binding partner under suitable conditions without any significant binding, for example, without any statistically significant binding, to other compounds present in a sample. In one aspect, the members of the binding pair have an affinity for each other that is at least about 50-, about 75-, or about 100-fold greater than the affinity between either member of the binding pair and other compounds present in the sample.

“Binding pair” refers to two molecules that specifically bind to each other. Examples of binding pairs include, but are not limited to, complementary oligonucleotides; antibody-antigen binding pairs; receptor-ligand binding pairs; enzyme-substrate binding pairs; hapten and hapten binding partner; and biotin and streptavidin or avidin. In one aspect, the binding pair includes an oligonucleotide sequence and a corresponding complementary oligonucleotide sequence that do not bind or cross-react with other oligonucleotide sequences present in a sample, composition or mixture under stringent conditions. In one aspect, the binding pair include an antigen-antibody pair or a receptor-ligand pair.

As used herein, the term “corresponding” refers to the relationship between members of a binding pair, for example, a capture reagent and an analyte or a targeting reagent and a targeting reagent complement. In one aspect, a member of a binding pair specifically binds to the “corresponding” member of the binding pair under suitable conditions without any significant binding, for example, without any statistically significant binding, to other components present in a sample or assay.

“Release” refers to delocalization of a previously collected material. In one aspect, the material is held at a localized position through chemical bonds or through specific or non-specific binding interactions. In another aspect, the material is held at a localized position by one or more physical barriers, such as a lipid-bilayer or plasma membrane. In one aspect, delocalization or release can be accomplished by breaking the bond or interaction so that the collected material can diffuse or mix into the surrounding media. In one aspect, delocalization or release can include physical delocalization of materials by disrupting a barrier, for example, disrupting a lipid bilayer or membrane, for example, using a lysis reagent so that the collected material can diffuse or mix into the surrounding media.

In the context of analytes measured in an assay, or a reagent used in an assay, the term “plurality” means more than one structurally and/or functionally different analyte or reagent (e.g., reagent A and reagent B), rather than just more than one copy of the analyte or reagent (e.g., reagent A and another copy of reagent A). For example, the term “plurality of detection reagents” means that more than one structurally or functionally different detection reagent is present in an assay, for example, the different detection reagents each specifically bind a different target analyte and does not describe a situation where there are multiple copies of one reagent. However, use of the term “plurality” in this context does not preclude the possibility that multiple copies are present of any of the plurality of analytes or reagents. For example, a plurality of immobilized targeting reagent complements could refer to immobilized targeting reagent complements that include one or more copies of targeting reagent complement A and one or more copies of targeting reagent complement B. When referring to a plurality of analytes or reagents, the terms “first,” “second,” “third,” etc. or “additional” can be used to distinguish between the unique analytes or reagents. For example, a “first” detection reagent binds to a “first” target analyte and a “second” detection reagent binds to a “second” target analyte or a different portion of the target analyte.

### III. Methods

In one aspect, a method of isolating, detecting or quantifying a surface marker displaying agent (SMDA) is provided. In one aspect, a method of isolating, detecting or quantifying a target analyte encapsulated by a surface marker displaying agent (SMDA) is provided. In one aspect, the method includes contacting a support surface with a sample that may include a SMDA in which a target analyte of interest is encapsulated. In one aspect, the method provides high-throughput isolation, detection, or quantification of a population of SMDA from a large number of samples in parallel. In one aspect, a method is provided for isolation, detection or quantification of a plurality of SMDA in a multiplexed assay. In one aspect, the method provides high-throughput isolation of one or more target analytes from one or more SMDA. In one aspect, a method is provided for isolating, detecting or quantifying a plurality of target analytes encapsulated by a SMDA. In one aspect, a method is provided for isolating, detecting or quantifying a plurality of target analytes encapsulated by a SMDA in a multiplexed assay.

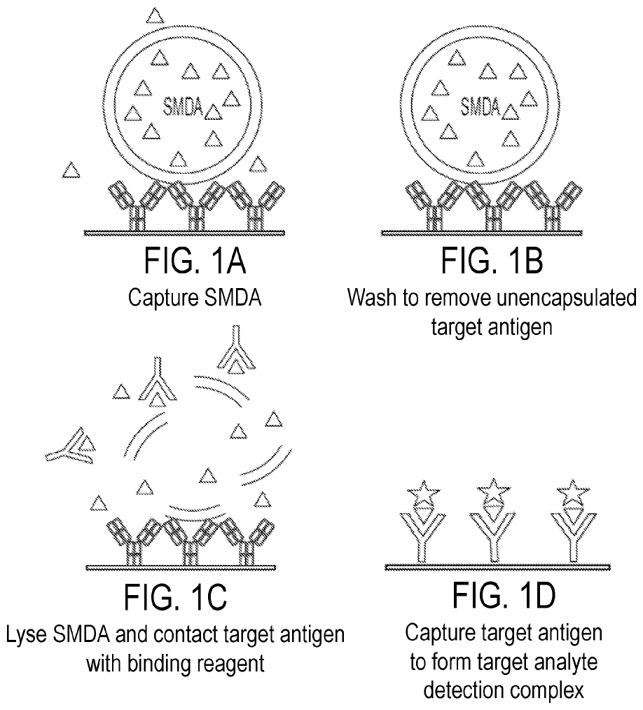

FIGS. 1A-1D provide a schematic illustration of a method for capturing a target analyte encapsulated in a SMDA. As shown in FIG. 1A, SMDA are captured on a support surface by selectively binding to a SMDA capture reagent that is immobilized on the support surface. In one aspect, the SMDA selectively binds, either directly or indirectly to a SMDA binding domain on the support surface. In one aspect, the SMDA selectively binds to a SMDA capture reagent immobilized on a SMDA binding domain of the support surface. In one aspect, the SMDA capture reagent selectively binds one or more surface markers present on the SMDA. In one aspect, one or more SMDA are releasably bound to the support surface. Methods for isolating SMDAs, including methods for multi-marker isolation, are described in International Application No. PCT/US2019/032995, filed May 17, 2019, entitled METHODS FOR ISOLATING SURFACE MARKER DISPLAYING AGENTS, the disclosure of which is hereby incorporated by reference in its entirety.

In one aspect, the SMDA capture reagent includes an SMDA capture reagent that is directly attached to the support surface. In one aspect, the SMDA capture reagent includes a targeting reagent that is capable of binding to a targeting reagent complement that is attached to the support surface. In one aspect, the SMDA capture reagent is immobilized on the support surface through a linker. In one aspect, the SMDA capture reagent is attached to the support surface through a pair of complementary oligonucleotides, in which one oligonucleotide is attached to the support surface and the other is attached to the SMDA capture reagent. In one aspect, the complementary oligonucleotides include a restriction site. In one aspect, the restriction site in the complementary oligonucleotides is cleaved by a restriction endonuclease to release the SMDA capture reagent from the support surface.

In one aspect, the SMDA capture reagent includes a targeting reagent that includes an oligonucleotide sequence that allows the SMDA capture reagent to be indirectly immobilized on the support surface. In one aspect, the SMDA capture reagent includes a targeting reagent that includes an oligonucleotide sequence that is complementary to a nucleotide sequence of a targeting reagent complement immobilized on the support surface. In one aspect, hybridization of the oligonucleotide targeting reagent to the oligonucleotide targeting reagent complement results in the immobilization of the SMDA capture reagent on the support surface. In one aspect, oligonucleotide targeting reagent includes a region complementary to the oligonucleotide targeting reagent complement that is at least about 10, about 11, about 12, about 13, about 14, about 15, about 20 or about 25 nucleotides in length and up to about 25, about 30 or about 35 nucleotides in length.

In one aspect, the sample includes target analyte that is encapsulated in the SMDA. In one aspect, the sample includes nonencapsulated target analyte. In one aspect, the nonencapsulated target analyte includes soluble target analyte present in the sample. In one aspect, the nonencapsulated target analyte is an unwanted component in the sample.

In one aspect, the method includes removing an unwanted component from the support surface after the SMDA is bound to the SMDA capture reagent. (See FIG. 1B). In one aspect, the method includes removing an unwanted component from the support surface after the SMDA immobilized on the support surface. In one aspect, the method includes a wash step for removing an unwanted component of the sample from the support surface. In one aspect, an unwanted component in the sample includes nonencapsulated target analyte. In one aspect, unwanted components are removed from the support surface with water or a wash buffer. Non-limiting examples of wash buffers include phosphate buffer, Tris buffer, HEPES buffer, and the like. In one aspect, the wash buffer includes a surfactant. In one aspect, the surfactant includes TWEEN-20.

In one aspect, the method includes contacting the sample with a blocking reagent to remove, destroy or block an unwanted component in the sample. In one aspect, the method includes contacting the sample with a blocking reagent to remove, destroy or block an unwanted component in the sample before the unwanted component is removed from the support surface. In one aspect, the method includes contacting the sample with a blocking reagent to remove, destroy or block an unwanted component in the sample before the wash step. In one aspect, the method includes contacting the sample with a blocking reagent to remove, destroy or block nonencapsulated target analyte in the sample. In one aspect, the blocking reagent binds to and prevents the nonencapsulated target analyte from binding to the target analyte binding domain on the support surface. In one aspect, the blocking reagent includes, but is not limited to, an antibody, antigen-binding antibody fragment or other antigen-binding reagent that specifically binds to the nonencapsulated target analyte. In one aspect, the blocking reagent includes, but is not limited to, an oligonucleotide or aptamer that specifically binds to the nonencapsulated target analyte. In one aspect, the blocking reagent includes an enzyme, including, but not limited to, a protease, nuclease or glycosidase that degrades the nonencapsulated target analyte.

In one aspect, the method includes contacting the support surface with a lysis reagent to lyse the SMDA. In one aspect, the SMDA is lysed to release encapsulated target analyte. In one aspect, the method includes releasing the immobilized SMDA from the support surface after the support surface is contacted with the lysis reagent. In one aspect, the method includes contacting the support surface with a lysis reagent after the SMDA is bound to the SMDA capture reagent. In one aspect, lysing the SMDA releases one or more encapsulated target analytes into the surrounding media. In one aspect, the lysis reagent includes a detergent. In one aspect, the lysis reagent includes TRITON X-100.

In one aspect, the method includes contacting the immobilized SMDA with a SMDA detection reagent to identify, detect or quantify the SMDA. In one aspect, the method includes contacting the immobilized SMDA with a SMDA detection reagent to identify, detect or quantify the SMDA before the support surface is contacted with the lysis reagent. In one aspect, the method includes contacting the immobilized SMDA with a SMDA detection reagent to identify, detect or quantify the SMDA after the support surface is contacted with the lysis reagent. In one aspect, the SMDA detection reagent specifically binds to a surface marker of the SMDA. In one aspect, the SMDA detection reagent binds to a surface marker of a SMDA that is bound to a SMDA capture reagent immobilized on a support surface. In one aspect, the SMDA detection reagent and surface marker remain bound to the immobilized SMDA capture reagent after the support surface is contacted with a lysis reagent. In one aspect, the SMDA detection reagent and surface marker remain bound to the immobilized SMDA capture reagent after the lysis reagent lyses the SMDA. In one aspect, the SMDA detection reagent includes a label. In one aspect, the SMDA detection reagent includes an antigen-binding substance, antigen, ligand, receptor, oligonucleotide, hapten, epitope, mimotope, or an aptamer. In one aspect, the SMDA detection reagent includes an antigen-binding substance that selectively binds a surface marker on the SMDA. In one aspect, the SMDA detection reagent includes an antigen-binding substance that selectively binds a disease-specific surface marker on the SMDA. In one aspect, the SMDA detection reagent includes an antigen-binding substance that selectively binds a cell-specific surface marker on the SMDA. In one aspect, the SMDA detection reagent includes an antibody or an antigen-binding antibody fragment. In one aspect, the SMDA detection reagent includes a labeled antibody or an antigen-binding antibody fragment. In one aspect, the SMDA detection reagent includes a labeled antibody or an antigen-binding antibody fragment that specifically binds to a surface marker of the SMDA.

In one aspect, the SMDA is released from the support surface before the target analyte is assayed. In one aspect, the SMDA is released from the support surface after lysis. In one aspect, the SMDA is released from the support surface by denaturing the binding between an oligonucleotide targeting reagent on a SMDA capture reagent and an oligonucleotide targeting reagent complement that is immobilized on the support surface.

In one aspect, the SMDA is released from the support surface by cleaving a chemical linker used to immobilize the SMDA to the support surface. There are many well-established cleavable chemical linkers that may be used that provide a covalent bond that may be cleaved without requiring harsh conditions. For example, disulfide containing linkers may be cleaved using thiols or other reducing agents, cis-diol containing linkers may be cleaved using periodate, metal-ligand interactions (such as nickel-histidine) may be cleaved by changing pH or introducing competing ligands. Similarly, there are many well-established reversible binding pairs that may be employed (including those that have been identified in the art of affinity chromatography). By way of example, the binding of many antibody-ligand pairs can be reversed through changes in pH, addition of protein denaturants or chaotropic agents, addition of competing ligands, etc. Other suitable reversible binding pairs include complementary nucleic acid sequences, the hybridization of which may be reversed under a variety of conditions including changing pH, decreasing salt concentration, increasing temperature above the melting temperature for the pair or adding nucleic acid denaturants (such as formamide).

In one aspect, shown in FIG. 1C, the method includes contacting the support surface with a target analyte capture reagent that selectively binds to the target analyte. In one aspect, the target analyte capture reagent includes a targeting reagent that is capable of selectively binding to a targeting reagent complement that is immobilized on a target analyte binding domain of the support surface. In one aspect, the support surface is contacted with the target analyte capture reagent after the SMDA is immobilized on the support surface. In one aspect, support surface is contacted with the target analyte capture reagent after the SMDA in the sample is immobilized on the support surface and after a wash step to remove unwanted components from the support surface. In one aspect, the unwanted components include nonencapsulated target analyte in the sample. In one aspect, as shown in FIG. 1C, a target analyte binding complex is formed in solution that includes a target analyte capture molecule and a target analyte.

In one aspect, the target analyte capture reagent is immobilized on a support surface. In one aspect, the target analyte capture reagent is immobilized in a binding domain on the support surface. In one aspect, the target analyte capture reagent is directly immobilized on a support surface. In one aspect, the target analyte capture reagent is indirectly immobilized on a support surface, for example, through the binding between a targeting reagent attached to the target analyte capture reagent and a targeting reagent complement that is immobilized on the support surface. In one aspect, the target analyte capture reagent is immobilized on a support surface before the SMDA is lysed. In one aspect, the target analyte capture reagent is immobilized on the support surface after the SMDA is lysed. In one aspect, the target analyte capture reagent is immobilized on a support surface before binding to the target analyte. In one aspect, the target analyte capture reagent is immobilized on the support surface after binding to the target analyte. In one aspect, the target analyte capture reagent binds to the target analyte to form a target analyte binding complex.

In one aspect, the SMDA is immobilized on a SMDA binding domain on a support surface and the immobilized SMDA is lysed to release encapsulated target analyte. In one aspect, the released target analyte is immobilized on a target analyte binding domain. In one aspect the support surface includes one or more SMDA binding domains and one or more target analyte binding domains. In one aspect, the support surface that includes one or more target analyte binding domains is the same as the support surface that includes one or more SMDA binding domains. In one aspect, the method includes contacting the released target analyte with a target analyte capture reagent to form a target analyte binding complex and eluting the target analyte binding complex from the support surface on which the SMDA is immobilized and contacting a support surface that includes one or more target analyte binding domains with the eluted target analyte binding complex. In one aspect, the target analyte capture reagent is immobilized on a bead such that the target analyte binding complex is formed on the bead and then eluted from the support surface on which the SMDA is immobilized.

In one aspect, the target analyte capture reagent includes a targeting reagent. In one aspect, the targeting reagent includes an antibody or antigen binding fragment thereof, antigen, ligand, receptor, oligonucleotide, hapten, epitope, mimitope, or an aptamer. In one aspect, the targeting reagent specifically binds to a targeting reagent complement that is immobilized on a support surface. In one aspect, the targeting reagent/targeting reagent complement binding pair includes antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, or aptamer/target molecule.

As shown in FIG. 1D, the target analyte is captured on a target analyte binding domain of the support surface where it can be detected. In one aspect, the target analyte is captured by selectively binding to a target analyte capture reagent that is immobilized on a target analyte binding domain of the support surface. In one aspect, allowing the target analyte to selectively bind to the target analyte capture reagent includes incubating the target analyte in the presence of the target analyte capture reagent under conditions suitable to allow selective binding. In one aspect, shown in FIG. 1D, a target analyte detection complex is formed on the support surface that includes the target analyte capture reagent, the target analyte, and the target analyte detection reagent.

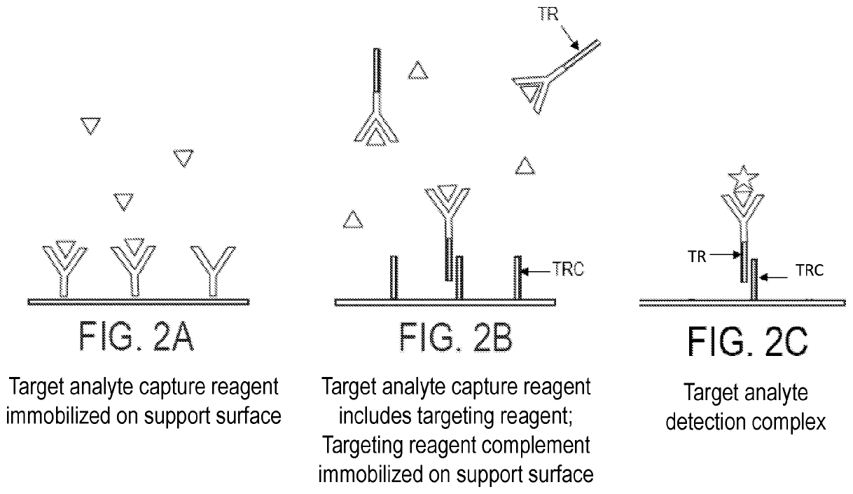

In a “direct immobilization” method, shown in FIG. 2A, the target analyte capture reagent is directly immobilized on the support surface.

In an “indirect immobilization” method, shown in FIG. 2B, the support surface is contacted with a target analyte capture reagent that can selectively bind the target analyte, wherein the target analyte capture reagent includes a targeting reagent (TR) that selectively binds to a targeting reagent complement (TRC) immobilized on the support surface. In one aspect, the target analyte capture reagent includes a targeting reagent (TR), and the target analyte binding domain includes a targeting reagent complement (TRC) that is a binding partner of the targeting reagent. In one aspect, the targeting reagent (TR) and the targeting reagent complement (TRC) include complementary oligonucleotide sequences. In one aspect, hybridization of the oligonucleotide targeting reagent to the oligonucleotide targeting reagent complement results in the immobilization of the target analyte capture reagent on the support surface. In one aspect, the oligonucleotide targeting reagent includes a region complementary to the oligonucleotide targeting reagent complement that is at least about 10, about 11, about 12, about 13, about 14, about 15, about 20 or about 25 nucleotides in length and up to about 25, about 30 or about 35 nucleotides in length. In one aspect, shown in FIG. 2B, a target analyte binding complex is formed in solution that includes the target analyte capture reagent and the target analyte. FIG. 2C illustrates a target analyte detection complex that is indirectly immobilized on the support surface, wherein the target analyte detection complex includes the target analyte capture reagent, the target analyte and a target analyte detection reagent that is immobilized through the binding of the targeting reagent (TR) to the targeting reagent complement (TRC).

In one aspect, the oligonucleotide targeting reagent includes a sequence that is complementary to a linker oligonucleotide. In one aspect, the linker oligonucleotide includes a nucleotide sequence that is complementary to a nucleotide sequence of the targeting reagent and a capture oligonucleotide that is immobilized on a support surface. In one aspect, hybridization of the linker oligonucleotide to the oligonucleotide targeting reagent and the capture oligonucleotide result in the indirect immobilization of the target analyte capture reagent to the support surface. In one aspect, the linker oligonucleotide includes a first region complementary to the oligonucleotide targeting reagent that is at least about 10, about 11, about 12, about 13, about 14, about 15, about 20 or about 25 nucleotides in length and up to about 25, about 30 or about 35 nucleotides in length and a second region complementary to the capture oligonucleotide that is at least about 10, about 11, about 12, about 13, about 14, about 15, about 20 or about 25 nucleotides in length and up to about 25, about 30 or about 35 nucleotides in length.

In one aspect, the method includes contacting the target analyte with a target analyte detection reagent that selectively binds the target analyte. In one aspect, the method includes contacting a target analyte with a target analyte detection reagent in solution, i.e., before the target analyte is immobilized on the support surface. In one aspect, the method includes contacting a target analyte with a target analyte capture reagent and a target analyte detection reagent in solution to form a target analyte detection complex. In one aspect, the method includes immobilizing the target analyte detection complex in a target analyte binding domain on a support surface. In one aspect, the method includes contacting a target analyte that is immobilized on a support surface with a target analyte detection agent. In one aspect, a target analyte detection complex is formed on a target analyte binding domain of the support surface that includes a target analyte capture reagent, the target analyte and a target analyte detection reagent. In one aspect, the target analyte detection reagent includes a detectable label.

In one aspect, the target analyte detection reagent includes a labeled antigen-binding substance, antigen, ligand, receptor, oligonucleotide, hapten, epitope, mimotope, or aptamer. In one aspect, the target analyte includes a nucleic acid sequence and the target analyte detection reagent includes a labeled complementary nucleic acid sequence that specifically binds to the nucleic acid sequence of the target analyte.

In one aspect, the target analyte is assayed while immobilized to the support surface. In one aspect, the target analyte is immobilized on a target analyte binding domain of the support surface. In one aspect, the support surface includes one or more SMDA binding domains and one or more target analyte binding domains. In one aspect, the target analyte is immobilized on one or more target analyte binding domains on a support surface that is separate from or different than the support surface that includes one or more SMDA binding domains. In one aspect, the target analyte is released from the support surface following removal of unwanted components from the surface. In one aspect, the target analyte is released from the support surface and further analyzed. In one aspect, the target analyte is eluted from the support surface while it is still bound to the target analyte capture reagent. In one aspect, the target analyte capture reagent is disassociated from the target analyte, for example, using a low-pH elution, before the target analyte is assayed.

In one aspect, the SMDA detection reagent or target analyte detection reagent include a nucleic acid probe. In one aspect, the method includes extending the nucleic acid probe of the detection reagent to form an extended sequence, wherein the extended sequence includes an oligonucleotide sequence that binds to an anchoring oligonucleotide immobilized on a binding domain of the support surface. In one aspect, the method includes detecting or determining an amount of extended sequence immobilized on the support surface.

In one aspect, the SMDA capture reagent or target analyte capture reagent include a primer oligonucleotide that generates an amplicon. In one aspect, the capture reagent includes an antibody or antigen binding fragment and a primer oligonucleotide that generates an amplicon. In one aspect, the capture reagent includes a primer oligonucleotide that contains a sequence that is complementary to an oligonucleotide template, such as a circular oligonucleotide. In one aspect, the primer is used to form an amplicon that includes a sequence complementary to a nucleotide sequence of an anchoring oligonucleotide. Any suitable amplification technique can be used to generate the amplicon, including but not limited to, PCR (Polymerase Chain Reaction), LCR (Ligase Chain Reaction), and isothermal amplification methods, e.g., helicase-dependent amplification, rolling circle amplification (RCA), 3SR (Self-Sustained Synthetic Reaction), transcription mediated amplification (TMA), nucleic acid sequence-based amplification (NASBA), signal mediated amplification of RNA technology, strand displacement amplification (SDA), loop-mediated isothermal amplification of DNA (LAMP), isothermal multiple displacement amplification, single primer isothermal amplification, and circular helicase-dependent amplification. In one aspect, the amplification technique is proximity ligation amplification (PLA) using RCA, for example, as described in International Appl. No. PCT/US2015/030925, published as WO 2015/175856, which is incorporated by reference in its entirety.

In one aspect, rolling circle amplification (RCA) is used to make the amplicon. RCA has significant advantages in terms of sensitivity, multiplexing, dynamic range and scalability. Techniques for RCA are known (see, e.g., Baner et al, Nucleic Acids Research, 26:5073 5078, 1998; Lizardi et al., Nature Genetics 19:226, 1998; Schweitzer et al. Proc. Natl. Acad. Sci. USA 97:10113 119, 2000; Faruqi et al., BMC Genomics 2:4, 2000; Nallur et al., Nucl. Acids Res. 29:e118, 2001; Dean et al. Genome Res. 11:1095 1099, 2001; Schweitzer et al., Nature Biotech. 20:359 365, 2002; U.S. Pat. Nos. 6,054,274, 6,291,187, 6,323,009, 6,344,329 and 6,368,801). Several different variants of RCA are known, including linear RCA (LRCA) and exponential RCA (ERCA). RCA generates many thousands of copies of a circular template, with the chain of copies attached to the original target DNA, allowing for spatial resolution of target and rapid amplification of the signal. RCA facilitates (i) detection of single target molecules; (ii) amplification of signals from proteins as well as DNA and RNA; (iii) identifying the location of molecules that have been amplified on a solid surface; (iv) measurement of many different targets simultaneously; and (v) analysis of one or more targets in solution or solid phase. The spatial localization of RCA products with the detection complex is especially advantageous when conducting multiplexed binding assays in an array or particle-based format.

In one aspect, the amplicon also includes one or more detection sequences and the measuring step includes contacting the amplicon with one or more labeled probes that are complementary to the one or more detection sequences of the amplicon. In one aspect, the amplicon remains localized on the surface following the amplification.

Methods for assaying target analytes, including multiplexed binding assays for a plurality of target analytes and various capture reagents are disclosed in U.S. Patent Publication No. 2016/0069872, filed Sep. 8, 2015, entitled METHODS FOR CONDUCTING MULTIPLEXED ASSAYS, the disclosure of which is incorporated by reference herein in its entirety. Multiplexed measurement of analytes on a surface including a plurality of binding domains using electrochemiluminescence has been used in the Meso Scale Diagnostics, LLC, MULTI-ARRAY® and SECTOR® Imager line of products (see, e.g., U.S. Pat. Nos. 10,201,812, 7,842,246 and 6,977,722, the disclosures of which are incorporated herein by reference in their entireties).

In one aspect, the target analyte is subjected to an ultrasensitive assay. Ultrasensitive assay formats for soluble proteins that marry a variation of proximity ligation amplification with ECL detection to provide state-of-the-art sensitivity for protein assays are described in International Appl. No. PCT/US2015/030925, published as WO 2015/175856. Such assays have detection limits as low as the pg/mL to sub pg/mL level and can detect as low as 1000 molecules per 25 uL sample.

In one aspect, the nucleic acid probe includes a targeting nucleic acid sequence that is complementary to at least a portion of a nucleic acid sequence of the target analyte and an extended nucleic acid sequence. In one aspect, the extended nucleic acid sequence includes a detection sequence complement that is complementary to a detection oligonucleotide. In one aspect, the amount of extended sequence immobilized on the support surface is measured by contacting the extended sequence with a detection oligonucleotide and measuring the amount of detection oligonucleotide immobilized on the support surface. In one aspect, the amount of extended sequence immobilized on the support surface is measured by contacting the extended sequence with a detection oligonucleotide and measuring the amount of detection oligonucleotide immobilized on the target analyte binding domain of the support surface.

In one aspect, the nucleic acid probe is extended to include an extended sequence. Any suitable amplification technique can be used to generate the extended sequence (or amplicon), including but not limited to, PCR (Polymerase Chain Reaction), LCR (Ligase Chain Reaction), and isothermal amplification methods, e.g., helicase-dependent amplification, rolling circle amplification (RCA), 3SR (Self-Sustained Synthetic Reaction), transcription mediated amplification (TMA), nucleic acid sequence-based amplification (NASBA), signal mediated amplification of RNA technology, strand displacement amplification (SDA), loop-mediated isothermal amplification of DNA (LAMP), isothermal multiple displacement amplification, single primer isothermal amplification, and circular helicase-dependent amplification. In one aspect, the amplification technique is proximity ligation amplification (PLA) using RCA, which is known in the art, and disclosed in International Appl. No. PCT/US2015/030925, published as WO 2015/175856, which is incorporated by reference in its entirety. In one aspect, the amplicon includes one or more detection sequences and the measuring step includes contacting the extended sequence with a detectably labeled oligonucleotide, wherein the detectably labeled oligonucleotide is complementary to a detection sequence of the amplicon. In one aspect, the amplicon includes one or more detection sequences and the measuring step includes contacting the extended sequence with one or more detectably labeled oligonucleotides complementary to the one or more detection sequences. In another aspect, the amplicon remains localized on the surface following amplification. In one aspect, detecting the target analyte includes measuring the amount of extended sequence immobilized on the support surface.

In one aspect, method is used in a singleplex format. In one aspect, the method is used in a multiplex format. In one aspect, multiple assay measurements are performed on a single sample. In a one aspect, the methods are used in a multiplexed format in which a plurality of target analytes bind to a plurality of target analyte capture reagents. Multiplex measurements that can be used include, but are not limited to, multiplex measurements i) that involve the use of multiple sensors; ii) that use discrete assay domains on a surface (e.g., an array) that are distinguishable based on location on the surface; iii) that involve the use of reagents coated on particles that are distinguishable based on a particle property such as size, shape, color, etc.; iv) that produce assay signals that are distinguishable based on optical properties (e.g., absorbance or emission spectrum); or v) that are based on temporal properties of assay signal (e.g., time, frequency or phase of a signal). In one aspect, a multiplex analysis uses a multi-well plate that includes a plurality of binding domains with a plurality of capture molecules which may capture different SMDA in one sample or different target analytes released from one or more SMDA.

In one aspect, two or more different populations of SMDAs can be isolated. In one aspect, two or more different SMDAs are immobilized on two different solid phases (e.g., different sets of beads or a planar substrate having multiple binding sites). In one aspect, a single sample is mixed with all of the solid phases in a single reaction capturing different populations of SMDA on each support surface. The different solid phases may be separated by physical properties (e.g., magnetism, size, or color), or the different target analytes may be eluted together. In one aspect, the different target analytes are distinguished using detectably labeled oligonucleotides as described herein.

In one aspect, an automated version of the methods described herein is provided using a high-throughput robotic liquid handling system. This system allows simultaneous preparation of up to 480 samples with accuracy and reproducibility unmatched by a human operator. In one aspect, the automated system is a free-standing, fully integrated system for carrying out immunoassays using ECL technology. This system, capable of simultaneously running up to five 96-well assay plates, including a robotic lab automation workstation for liquid handling and plate manipulation, physically integrated with an ECL reader.

### A. Samples

In one aspect, one or more SMDA are isolated from a sample. In one aspect, the sample includes one or more SMDA and one or more unwanted components. In one aspect, the sample is purified before the sample is contacted with a support surface. In one aspect, the sample is purified by precipitation, differential centrifugation, ultracentrifugation, ultrafiltration, size-exclusion chromatography, affinity purification, including immuno-affinity purification, or a combination thereof. In one aspect, the affinity purification may be performed with magnetic or non-magnetic beads. In one aspect, the sample includes purified SMDA. In one aspect, the sample includes purified EVs.

Biological samples that may be analyzed include, but are not limited to, physiological samples or samples containing suspensions of cells, such as mucosal swabs, tissue aspirates, tissue homogenates, cell cultures, and cell culture supernatant, including cultures of eukaryotic and prokaryotic cells. In one aspect, cells are removed from the sample, for example, by centrifugation or filtration, before the support surface is contacted with the SMDA.

In one aspect, the sample is a mammalian fluid, secretion, or excretion. In one aspect, the sample is a purified mammalian fluid, secretion, or excretion. In one aspect, the sample is a sample of tissue or bodily fluid, for example, blood. In one aspect, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions. In one aspect, the sample is cerebrospinal fluid. In one aspect, the sample is a tissue or bodily fluid sample that includes a mixture of cancer and non-cancer cells.

In one aspect, the sample is an environmental sample. In one aspect, the sample is a bacterial sample. In another aspect, the sample is a mixed bacterial sample.

In one aspect, the sample includes EVs produced from a cell differentiated from a cell-line, differentiated from an induced pluripotent stem cell, a primary cell, or a combination thereof. Samples further include cell supernatants, such as those from neuronal and astrocyte cultures, which include at least the following: human cortical neurons differentiated from induced pluripotent stem cells (iPSC) and from the HCN-2 cell line, as well as mature astrocytes differentiated from iPSC and primary human astrocytes. In one aspect, samples include supernatants from oligodendrocytes derived from iPSC cells, which are commercially available, and from cell lines such as HOG or M03.13 which can be differentiated to mature oligodendrocytes using established protocols. Samples further include iPSC derived microglia, which are commercially available, as well as primary microglia which can be expanded in culture. Non-limiting examples of cell lines include MOLT-4 (differentiated or undifferentiated), Jurkat, HL60 (differentiated or undifferentiated), U-937 (differentiated or undifferentiated), HDLM-2, THP-1 (differentiated or undifferentiated), GA10, Ramos, HUVEC, PANC-1, Expi293, HaCat, HCT-15, H-2228, peripheral blood mononuclear cells (PBMCs), KU-812, MC-04, HT-1376, TT, HCT-1116, MCF-7, Calu-3, and the like.

In one aspect, the samples include EVs produced from a T cell, a B cell, a dendritic cell, an NK cell, a monocyte, a macrophage, a granulocyte, a platelet, an erythrocyte, an endothelial cell, an epithelial cell, a stem cell precursor cell, a mesenchymal stem cell, a leukocyte, or a senescent cell. T cells include, e.g., helper T cells, such as the subtypes Th1, Th2, Th9, Th17, Th22, and Tfh; regulatory T cells; killer T cells; γδ TCR+ T cells; and natural killer T cells.

In one aspect, the sample includes one or more viral particles.

### B. Surface Marker Displaying Agents

“Surface Marker Displaying Agent” (SMDA) refers to any membrane-bound structure in which one or more surface markers or biomarkers are present on the surface. “Membrane” refers to a boundary layer separating an interior vesicle space from an exterior space that includes one or more biological molecules such as lipids, and in some instances, carbohydrates, polypeptides or polysaccharides such as glycans. Membranes can include lipids or fatty acids, including, for example, phospholipids, phosphatidylserine, sphingolipids, sterols, glycolipids, fatty acids, cholesterols, or phosphoglycerides.

In one aspect, the SMDA includes a surface such as a lipid bilayer, membrane, cell wall, or envelope on which one or more surface markers are displayed. A surface maker is not limited to a marker physically on the surface of the cell and may include, but is not limited to surface antigens, transmembrane receptors or co-receptors, macromolecules immobilized on the surface, such as aggregated proteins or carbohydrates, internal cellular components and the like. SMDA include naturally occurring, partially synthetic, or fully synthetic agents. In one aspect, the SMDA is a biologically relevant material or component.

SMDA can have one or more types of cargo encapsulated therein, including, but not limited to, proteins, nucleic acids, such as DNA, RNA, including miRNA, lipids, carbohydrates, small molecules such as hormones, cofactors, vitamins, minerals, salts, metals, metal-containing compounds, a metabolite, or combinations thereof.

Examples of surface marker displaying agents include cells, viruses and viral particles, cellular organelles, vesicles, and extracellular vesicles (EVs) or exosomes.

In one aspect, the SMDA is a cell. In one aspect, the SMDA is a prokaryotic or eukaryotic cell. In one aspect, the cell is a bacterial cell. In one aspect, the cell is an archaeal cell. In one aspect, the cell is a eukaryotic cell. In one aspect, the cell is a mammalian cell. In one aspect, the cell is an animal cell. In one aspect, the cell is a human cell. In one aspect, the cell is an insect cell. In one aspect, the cell is a plant cell. In one aspect, the cell is a yeast cell. In one aspect, the cell is a bacterial cell. In one aspect, the cell is a variant of a particular cell type, for example, an abnormal cell, such as a cancer cell

In one aspect, the SMDA is a virus or viral particle. In one aspect, the virus or viral particle includes a capsid that includes one or more capsid proteins or viral coat proteins. In one aspect, the viral or viral particle includes a viral envelope. In one aspect, the viral envelope includes a lipid bilayer and one or more viral proteins. In one aspect, the viral particle includes one or more structural proteins that include, but are not limited to, a spike protein, a nucleocapsid protein, a membrane protein or an envelope protein. In one aspect, the virus is a DNA or RNA virus, including, for example, a single stranded (ss) or double stranded (ds) DNA or RNA virus. In one aspect, the virus is an enveloped or a non-enveloped virus. Enveloped viruses include, but are not limited to herpesvirus, poxvirus, orthomyxovirus, paramyxovirus, rhabdovirus, filovirus, bunyavirus, arenavirus, coronavirus, retrovirus, and influenza virus. Non-enveloped viruses include, but are not limited to, norovirus, rhinovirus and poliovirus. In one aspect, the enveloped virus is a coronavirus. In one aspect, the enveloped virus is SARS-CoV-2.

In one aspect, the SMDA is a cellular organelle, including, but not limited to the Golgi apparatus, mitochondria, endoplasmic reticulum, nucleus, vacuoles or chloroplasts.

In one aspect, the SMDA is a vesicle. In one aspect, the vesicle is a lysosome, an endosome, a peroxisome or a liposome.

In one aspect, the vesicle is an extracellular vesicle. “Extracellular vesicle” or EV refers a cell-derived vesicle having a membrane that surrounds and encloses a central internal space. EVs are a class of membrane bound organelles secreted by various cell types for intercellular communication and for the transfer of genetic materials, proteins, lipids, and metabolites. EV refers to a broad spectrum of vesicles secreted by different types of cells and includes exosomes, ectosomes, oncosomes, shed vesicles, microvesicles, and apoptotic bodies. EVs are defined by the International Society of Extracellular Vesicles (see Gardiner et al., (2016) Journal of Extracellular Vesicles 5(1):32945).

EV membranes can include a lipid bi-layer with an external surface and an internal surface bounding an enclosed volume. In one aspect, the EV has a cross-sectional diameter smaller than the cell from which they are secreted, for example, from about 10 nm, 20 nm, 30 nm, 40 nm or 50 nm and up to about 100 nm, 200 nm, 300 nm, 400 nm, 500 nm or 1000 nm.

In one aspect, the EV of interest is secreted from a cell of the central nervous system (CNS). In one aspect, the cell of the CNS is a neuron, an astrocyte, an oligodendrocyte or microglia.

In one aspect, the EV is an exosome, a micro-vesicle or a large-oncosome.

### C. Surface Marker

In one aspect, the surface marker is common to EVs. Such surface markers include, but are not limited e.g., tetraspanins, such as CD9, CD37, CD63, CD81, CD82. In one aspect, the surface marker is specific to a CNS EV. In one aspect, the surface marker is specific to a neuron EV, an astrocyte EV, an oligodendrocyte EV or a microglia EV.

In one aspect, the surface marker is a lysosome-specific marker. Examples of lysosome-specific markers include, e.g., LAMP1, LC3, and ATG5. In one aspect, the surface marker is an endosome-specific marker. Examples of endosome-specific markers include, e.g., EEA1, Rab5, Rab7, and palladin. In one aspect, the surface marker is a peroxisome-specific marker. An example of a peroxisome-specific marker is catalase. In one aspect, the vesicle is a liposome. Liposomes can be artificial vesicles that include engineered surface markers.

In one aspect, the surface marker is a surface adhesion protein. Exemplary surface adhesion proteins include, but are not limited to, EpCAM, E-Cadherin, P-Cadherin, LICAM, NCAM1, Nectin-4, PECAM and ICAM-1. In one aspect, the surface marker is a surface receptor. Exemplary surface receptors include, but are not limited to, EGFR, EphA2, TFRC, FasR, and TNFR1. In one aspect, the surface marker is an endothelial marker. Exemplary endothelial markers include, but are not limited to, PECAM, CD276, TEM7, TEM8, and thrombomodulin.

In one aspect, the surface marker is a viral protein. In one aspect, the surface marker is a viral coat protein. In one aspect, the surface marker is a capsid protein. In one aspect, the surface marker is a viral envelope protein. In one aspect, the surface marker is a viral protein, for example, a spike protein, a nucleocapsid protein, a membrane protein or an envelope protein.

In one aspect, at least one surface marker is a central nervous system (CNS) cell marker. In one aspect, the surface marker is specific to a neuron, an astrocyte, an oligodendrocyte or microglia. In one aspect, the surface marker is specific to a neuron. In one aspect, the surface marker specific to a neuron is L1CAM, NCAM, NRCAM, CHL1, Glu-R2, neurofascin, DAT1, CD90, CD24 or synaptophysin. In one aspect, the neuron is a dopaminergic neuron, a GABAergic neuron, a cholinergic neuron, a serotonergic neuron or a glutamatergic neuron.

In one aspect, the surface marker is specific to an astrocyte. In one aspect, the surface marker specific to an astrocyte is ALDH1L1, GLT-1, GLAST, CD184, CD44, A2B5, CD80 or CD86. In one aspect, the surface marker is specific to an oligodendrocyte. In one aspect, the surface marker specific to an oligodendrocyte is O4, PDGFRa, CSPG4, GD3, MOG, or MBP. In one aspect, the surface marker is specific to microglia. In one aspect, the microglia surface marker is Tmem119, CD11bF4/80, CD68, P2RY12, CXC3R1. In one aspect, the surface marker is a disease-specific biomarker.

In one aspect, the surface marker is specific to a T cell, a B cell, a dendritic cell, an NK cell, a monocyte, a macrophage, a granulocyte, a platelet, an erythrocyte, an endothelial cell, an epithelial cell, a stem cell precursor cell, a mesenchymal stem cell, a leukocyte, a T lymphocyte, or a B lymphocyte. T cells include, e.g., helper T cells, such as the subtypes Th1, Th2, Th9, Th17, Th22, and Tfh; regulatory T cells; killer T cells; γδ TCR+ T cells; and natural killer T cells.

In one aspect, the surface marker is specific to a T cell, a helper T cell, a regulatory T cell, a killer T cell, a γδ TCR+ T cell, or a natural killer T cell. In one aspect, the surface marker specific to a T cell is CD3, CD4, CD8 or CD2. In one aspect, the surface marker specific to a helper T cell is CD5, CD6, CD45, CD62L, CD197 (CCR7), or a/b TCR. In one aspect, the surface marker specific to a helper T cell subtype Th1 is CD183 (CXCR3), CD119 (IFNy Ra), CD195 (CCR5), CD218a (IL-18Ra), LT-BR, or CD336 (TIM-3). In one aspect, the surface marker specific to a helper T cell subtype Th2 is CD194 (CCR4), Crth2, CDw198 (CCR8), CRTH2, IL33-Ra, or CD365 (TIM-1). In one aspect, the surface marker specific to a helper T cell subtype Th17 is CD196 (CCR6), CD161, or IL-23R. In one aspect, the surface marker specific to a helper T cell subtype Th22 is CCR10. In one aspect, the surface marker specific to a helper T cell subtype Tfh is CD185 (CXCR5), CD84, CD126 (IL-6Ra), CD150, CD154, CD252 (OX40L), CD278 (ICOS), or CD279 (PD1). In one aspect, the surface marker specific to a regulatory T cell is CD25, CD39, CD73, CD103, CD152 (CTLA-4), GARP, or GITR. In one aspect, the surface marker specific to a killer T cell is CD8. In one aspect, the surface marker specific to a γδ TCR+ T cell is γδ TCR. In one aspect, the surface marker specific to a natural killer T cell is CD56 (NCAM), CD11b, CD11c, CD16, CD32, CD49b, CD57, CD69, CD94, CD122, CD158, CD161 (NK1.1), CD244, CD314, CD319, CD328, CD355, Ly49, Ly108, or Vα24-Jα18 TCR.

In one aspect, the surface marker is specific to a B cell. In one aspect, the surface marker specific to the B cell is CD19, CD20, CD5, CD9, CDIIa, CD18, CD25, CD26, CD29, CD31, CD38, CD44, CD45, CD49b, CD49c, CD49d, CD50, CD54, CD58, CD62L, CD73, CD95, CD102, CD119, CD120a, CD120b, CD124, or CD166.

In one aspect, the surface marker is specific to a dendritic cell. In one aspect, the surface marker specific to the dendritic cell is CD11c, CD123, CDIa, CD33, CD45, CD49d, CD49e, CD58, CD73, CD120a, CD120b, CD123, or CD271.

In one aspect, the surface marker is specific to a NK cell. In one aspect, the surface marker specific to the NK cell is CD56, CDIIa, CD18, CD25, CD26, CD29, CD31, CD38, CD45, CD49b, CD49d, CD49e, CD50, CD58, CD59, CD62L, CD95, CD119, CD120a, CD120b, or CD178.

In one aspect, the surface marker is specific to a monocyte or a macrophage. In one aspect, the surface marker specific to the monocyte or macrophage is CD14, CD33, CD4, CD9, CDIIa, CD13, CD15, CD18, CD26, CD29, CD31, CD38, CD44, CD45, CD49a, CD49b, CD49c, CD49e, CD49f, CD50, CD51, CD54, CD58, CD59, CD61, CD62L, CD63, CD95, CD102, CD119, CD120a, CD120b, CD123, CD124, or CD127.

In one aspect, the surface marker is specific to a granulocyte. In one aspect, the surface marker specific to the granulocyte is CD66b, CD4, CD9, CDIIa, CD13, CD14, CD15, CD18, CD29, CD31, CD33, CD44, CD45, CD50, CD58, CD59, CD63, CD95, CD119, CD120a, CD120b, CD123, or CD178.

In one aspect, the surface marker is specific to a platelet. In one aspect, the surface marker specific to the platelet is P-selectin, PECAM, CD41, CD61, CD62, CD9, CD29, CD31, CD44, CD49b, CD49f, CD51, CD63, CD102, CD120a, CD120b, or CD140a.

In one aspect, the surface marker is specific to an erythrocyte. In one aspect, the surface marker specific to the erythrocyte is CD235a, CD49e, CD58, CD59, CD49e, CD58, or CD235a.

In one aspect, the surface marker is specific to an endothelial cell. In one aspect, the surface marker specific to the endothelial cell is CD146. In one aspect, the surface marker is specific to an epithelial cell. In one aspect, the surface marker specific to the epithelial cell is CD326. In one aspect, the surface marker specific to the endothelial or epithelial cell is CD9, CD10, CD13, CD26, CD29, CD31, CD34, CD49b, CD49c, CD49d, CD49e, CD49f, CD50, CD51, CD54, CD58, CD61, CD62E, CD62P, CD63, CD71, CD90, CD102, CD104, CD105, CD109, CD119, CD120a, CD120b, CD121a, CD123, CD124, CD133, CD140a, CD140b, CD144, CD146, CD166, or CD178.

In one aspect, the surface marker is a cancer antigen. In one aspect, the surface marker is a tumor antigen. In one aspect, the cancer antigen is 5′-nucleotidase (CD73), B7-H3 (CD276), CA19.9, CA50, CA60, cadherin-1 (CD324), CD44v6, ADAM10 (CD156c), basigin (CD147), CD24, CD91, Cripto-1 (TSGF1), E-selectin (CD62e), FLT-3 ligand, A1CAM (CD166), Claudin-3, Claudin-4, EGFR, EGFRvIII, CDCP1 (CD318), CEACAM5 (CD66e), Ephrin receptor A2, Glypican-1, HIST2H2BE, HIST2H2BF, CD44, Galectin-3-binding-protein, MAGE3/6, Gamma-enolase (NSE), IL-2R, KIT (CD117), KNG2DL2 (ULBP-2), EpCAM (CD326), FasR (CD95), FasL, HER-2, ICAM-1 (CD54), Integrin A6 (CD49f), Integrin B4 (CD104), Mucin-4, Prominin-1 (CD133), Wnt-2, Mucin-16, Mucin-18 (CD146), Sialyl Lewis X, Syndecan-1 (CD138), TNFR1 (CD120a), upaR (CD87), L1CAM (CD171), MET, MUC1 (CA15-3), Raph Blood group (CD151), Tspan8, EphB4, CEA, ALCAM (CD166), DCC (netrin 1 receptor), LRIG3, Nectin-4, TNFSF8, YES, Galectin-9, Vimentin, or Cytokeratin. Exemplary tumor antigens include, but are not limited to, CEA, CA19.9, CA50, CA125, CA15.3, mesothelin, cytokeratin-8, E-cadherin, EGFR, EpCAM, EphA2, NCAM, P-cadherin, cMET, Flt-3L, TNFR-2, cKit, ErbB2, and ANXA1. In one aspect, the tumor antigen markers are pancreatic cancer markers.

In one aspect, the surface marker is specific to a tumor infiltrating leukocyte. In one aspect, the surface marker specific to a tumor infiltrating leukocyte is LAG-3, TIM-3, PD-1 (CD279), CD44, PD-L1, CTLA-4, or CD28. In one aspect, the surface marker is an antigen presenting cell marker. In one aspect, the antigen presenting cell marker is CD80, CD86, or CD83. In one aspect, the surface marker is an immuno-oncology marker. In one aspect, the immune-oncology marker is CD137, CD154, or CD40.

In one aspect, the surface marker is specific to a stem cell. In one aspect, the surface marker specific to a stem cell is ABCG2 (CD338), CD9, CD11b, CD20, CD29, CD31, CD34, CD44, CD45, CD49f, CD56, CD73, CD81, CD90, CD95, CD105, CD117, CD118, CD133, CD144, CD146, CD166, CD184, DLK1, STRO-1, TNAP, CD24, SSEA-3, SSEA-4, TRA-1-60, or TRA-1-81. In one aspect, the surface marker is specific to a mesenchymal stem cell. In one aspect, the surface marker specific to a mesenchymal stem cell is CD73, CD105, CD90, CD29, CD44, CD166, CD13, CD14.

In one aspect, the surface marker is a cell adhesion molecule, an integrin, a classical cadherin, a desmosomal cadherin, a protocadherin, an unconventional cadherin, a claudin, or a selectin. In one aspect, the cell adhesion molecule is ICAM1, ICAM2, ICAM3, ICAM4, ICAM5, VCAM1, PECAM-1, NCAM, or ALCAM. In one aspect, the integrin is VLA-1, VLA-2, VLA-3, VLA-4, VLA-5, VLA-6, LFA-1, MAC-1, CD11c/CD18, CD41/CD61, vitronectin-R, or CD49d. In one aspect, the classical cadherin is CDH1, CHD2, CDH12, or CDH3. In one aspect, the desmosomal cadherin is DSG1, DSG2, DSG3, DSG4, DSC1, DSC2, or DSC3. In one aspect, the unconventional cadherin is CDH4, CDH5, CDH6, CDH7, CDH8, CDH9, CDH10, CDH11, CDH13, CDH15, CDH16, CDH17, CDH18, CDH19, CDH20, CDH21, CDH22, CDH23, CDH24, CDH26, CDH28. In one aspect, the claudin is CDLN1, CDLN2, CDLN3, CDLN4, CDLN5, CDLN6, CDLN7, CDLN8, CDLN9, CDLN10, CDLN11, CDLN12, CDLN13, CDLN14, CDLN15, CDLN16, CDLN17, CDLN18, CDLN19, CDLN20, CDLN21, CDLN22, CDLN23 or CDLN24. In one aspect, the selectin is E-selectin, P-selectin, or L-selectin.

In one aspect, the surface marker is specific to a senescent cell. In one aspect, the surface marker specific to the senescent cell is DPP4, CD26, CD57, or CD16.

### D. Detecting the SMDA

In one aspect, the SMDA is assayed. In one aspect, visualization or quantification of the SMDA is performed prior to releasing the SMDA from the support surface. In one aspect, visualization or quantification of the SMDA is performed prior to lysing the SMDA. In one aspect, visualization or quantification of the SMDA is performed after lysing the SMDA. In one aspect, visualization or quantification of the target analyte is performed after the SMDA is released from the support surface. In one aspect, the SMDA is detected using a detectable label, for example, an electrochemiluminescent or fluorescent label.

### E. Target Analyte

In one aspect, one or more SMDA in a sample encapsulate one or more target analytes of interest. Target analytes include, but are not limited to, proteins, nucleic acids, lipids, carbohydrates, small molecules such as hormones, cofactors, vitamins, minerals, salts, metals, metal-containing compounds, a metabolite, or combinations thereof. In one aspect, the method includes conducting an assay to detect, quantitate, characterize, or a combination thereof, an encapsulated target analyte. In one aspect, the assay is an ultrasensitive assay.

Databases are available that characterize the content of SMDAs. Three databases are publicly accessible: Exocarta, Vesiclepedia, and EVpedia (Kim et al., (2013) EVpedia: an integrated database of high-throughput data for systemic analyses of extracellular vesicles, J Extracell Vesicles 2:1-7; Kalra et al., (2012) Vesiclepedia: a compendium for extracellular vesicles with continuous community annotation, PLOS Biol.; Mathivanan S and Simpson RJ. (2009) ExoCarta: a compendium of exosomal proteins and RNA, Proteomics 9:4997-5000; Simpson et al., (2012) ExoCarta as a resource for exosomal research, J Extracell Vesicles; Mathivanan et al., (2012) ExoCarta: database of exosomal proteins RNA and lipids, Nucleic Acids Res. doi: 10.1093/nar/gkr828), each of which is incorporated by reference in its entirety. All databases include the protein, nucleic acid, and lipid content together with the isolation and purification procedures used to generate the data.

Exemplary target analytes include, but are not limited to, TSG101, HSP70, ALIX/PDCD6IP, Flotillin, Tuj1, Tyr hydroxylase, NSE, NF-L, GFAP, S100β, GluSyn, CNPase, Oligo2, TMEM119, or a combination thereof.

In one aspect, the target analyte is a viral protein. In one aspect, the target analyte is a structural viral protein. In one aspect, the target analyte is a viral capsid protein. In one aspect, the target analyte is a viral nucleocapsid protein. In one aspect, the target analyte is a viral genomic sequence. In one aspect, the target analyte is a viral DNA or RNA sequence. In one aspect, the target analyte is a mutant viral DNA or RNA sequence.

### F. Unwanted Components

In one aspect, the method includes removing unwanted components of the sample or unwanted components of the SMDA from the support surface. In one aspect, unwanted components in the sample are removed from the support surface after the SMDA is immobilized on the support surface, for example, using a wash solution.

In one aspect, unwanted components in the sample include, but are not limited to components that do not specifically bind to a SMDA capture reagent or a target analyte capture reagent on the support surface. In one aspect, an unwanted component is a component that may be present in the sample that is not a SMDA in which a target analyte is encapsulated, or a buffer, solution or other reagent used in the method described herein. In one aspect, the unwanted component in the sample interferes with the results of the method, for example, by producing a false positive or false negative result. Examples of unwanted components include, but are not limited to, biomolecules, such as proteins, lipoproteins, or nucleic acids; and chemical impurities, such as salts or other reagents, for example, reagents used in isolating the SMDA from a biological sample. In one aspect, an unwanted component in the sample includes nonencapsulated target analyte, including, for example, a soluble form of the analyte present in the sample. In one aspect, an unwanted component in the sample includes SMDA that do not bind to the capture reagent, for example, SMDA that do not have a surface marker to which the SMDA capture reagent binds. In one aspect, unwanted components are soluble in the washing fluid.

In one aspect, the method includes lysing the SMDA to release the target analyte and capturing the released target analyte on the support surface. In one aspect, unwanted components of the SMDA are removed from the support surface after the target analyte is immobilized on the support surface, for example, using a wash solution. In one aspect, unwanted components of the SMDA include, but are not limited to, components of the lysed SMDA that do not selectively bind to a SMDA or target analyte capture reagent on the support surface.

### G. Support Surface

In one aspect, a support surface is contacted with a sample including a SMDA encapsulating one or more target analytes of interest. The term “contacting” has its ordinary meaning to one of skill in the art. Methods of contacting samples, e.g., liquids, solids, gels, etc., are known to those of ordinary skill in the art. In one aspect, the support surface includes one or more binding domains. In one aspect, the support surface includes one or more capture reagents immobilized on one or more binding domains. In one aspect, the support surface includes one or more SMDA capture reagents immobilized on one or more SMDA binding domains. In one aspect, the support surface includes one or more target analyte capture reagents immobilized on one or more target analyte binding domains. In one aspect, one or more SMDA capture reagents are immobilized on the same support surface as one or more target analyte capture reagents. In one aspect, the support surface includes one or more SMDA capture reagents immobilized on one or more SMDA binding domains and one or more target analyte capture reagents immobilized on one or more target analyte binding domains. In one aspect, one or more SMDA capture reagents are immobilized on a support surface that is separate from or different than the support surface on which one or more target analyte capture reagents are immobilized. In one aspect, the support surface includes one or more targeting reagent complements immobilized on one or more binding domains. In one aspect, the support surface includes one or more targeting reagent complements immobilized on one or more binding domains, wherein the targeting reagent complements hybridize to a targeting reagent of a SMDA capture reagent. In one aspect, the support surface includes one or more targeting reagent complements immobilized on one or more binding domains, wherein the targeting reagent complement hybridizes to a targeting reagent of a target analyte capture reagent. In one aspect, the binding domains are elements of an array.

Suitable support surfaces for use in the methods described herein are known in the art, and include conventional surfaces used in binding assays. Suitable support surfaces are disclosed, for example, in International Appl. No. PCT/US2015/030925, published as WO 2015/175856. Support surfaces may be made from a variety of different materials including polymers (e.g., polystyrene and polypropylene), ceramics, glass, composite materials (e.g., carbon-polymer composites such as carbon-based inks). Suitable support surfaces include surfaces of macroscopic objects such as an interior surface of an assay container (e.g., test tubes, cuvettes, flow cells, FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc.

In one aspect, the support surface includes a single component, for example, a multi-well plate. In one aspect, the support surface is a 96-well plate. In one aspect, the method uses a high throughput 96-well plate format in combination with a single immunoaffinity capture step. In one aspect, the support surface includes multiple components, including, for example, particles, such as beads. In one aspect, the support surface defines one or more boundaries of a container (e.g., a flow cell, well, cuvette, etc.) which holds the sample or through which the sample is passed.

In one aspect, the support surface includes multiple components, for example, particles or beads. Suitable support surfaces include particles (including but not limited to colloids or beads) commonly used in other types of particle-based assays, including, but not limited to, magnetic, polypropylene, and latex particles, hydrogels, such as agarose, materials typically used in solid-phase synthesis such as polystyrene and polyacrylamide particles, and materials typically used in chromatographic applications such as silica, alumina, polyacrylamide, polystyrene. The materials may also be a fiber such as a carbon fibril. Microparticles may be inanimate or alternatively, may include animate biological entities such as cells, viruses, bacterium and the like. A particle used in the present method may include any material suitable for attachment to one or more capture or anchoring reagents, and that may be collected via, e.g., centrifugation, gravity, filtration or magnetic collection. A variety of different types of particles to which capture reagents may be attached are sold commercially for use in binding assays. These include non-magnetic particles as well as particles including magnetizable materials which allow the particles to be collected with a magnetic field. In one aspect, the particles include a conductive or semiconductive material, e.g., colloidal gold particles. The microparticles may have a wide variety of sizes and shapes. By way of example and not limitation, microparticles may be between 5 nanometers and 100 micrometers. In one aspect, microparticles have sizes between 20 nm and 10 micrometers. The particles may be spherical, oblong, rod-like, etc., or they may be irregular in shape.

The particles used in the present method may be coded to allow for the identification of specific particles or subpopulations of particles in a mixture of particles. The use of such coded particles has been used to enable multiplexing of assays employing particles as solid phase supports for binding assays. In one approach, particles are manufactured to include one or more fluorescent dyes and specific populations of particles are identified based on the intensity or relative intensity of fluorescence emissions at one or more wave lengths. This approach has been used in the Luminex xMAP systems (see, e.g., U.S. Pat. No. 6,939,720) and the Becton Dickinson Cytometric Bead Array systems. Alternatively, particles may be coded through differences in other physical properties such as size, shape, imbedded optical patterns and the like. One or more particles provided in a mixture or set of particles may be coded to be distinguishable from other particles in the mixture by virtue of particle optical properties, size, shape, imbedded optical patterns and the like.

In one aspect, one or more capture reagents are immobilized on discrete binding domains on a support surface. In one aspect, one or more capture reagents are immobilized on a support surface by covalent bonds between the capture reagent and the support surface. In one aspect, one or more capture reagents are immobilized on a support surface by non-covalent interactions between the capture reagent and the support surface. In one aspect, one or more capture reagents are immobilized in one or more discrete binding domains in a binding array wherein the binding domains are individual array elements. In one aspect, one or more capture reagents are immobilized on one or more particles or beads wherein the binding domains are the individual beads. In one aspect, discrete assay signals are generated on and measured from each binding domain. In one aspect, capture reagents for different target analytes are immobilized in different binding domains such that the target analytes immobilized on each domain can be measured independently. In one example, the binding domains are prepared by immobilizing, on one or more surfaces, one or more discrete domains of capture reagents that bind one or more target analytes of interest.

In one aspect, a SMDA capture reagent that selectively binds a SMDA is immobilized on the support surface. In one aspect, a SMDA capture reagent is immobilized on a SMDA binding domain on the support surface. In one aspect, a target analyte capture reagent that selectively binds a target analyte is immobilized on the support surface. In one aspect, the target analyte capture reagent is immobilized in a target reagent binding domain on the support surface.

In one aspect, one or more capture reagents are releasably bound to the support surface. In one aspect, one or more capture reagents are releasably bound to the support surface by a labile linker. In one aspect, a SMDA capture reagent is releasably bound to the support surface. In one aspect, a target analyte capture reagent is releasably bound to the support surface. In one aspect, the labile linker is a heat-labile, a photolabile, or a chemically labile linker. In another aspect, the labile linker includes an oligonucleotide that is complementary to an oligonucleotide immobilized on the surface or is an oligonucleotide including a restriction site cleavable by a restriction endonuclease. In one aspect, the labile linker is a small molecule that binds to a protein on the surface. In one aspect, the capture reagent is biotinylated, and the surface is coated with streptavidin. In one aspect, the surface is an MSD plate electrode or a particle.

In one aspect, the SMDA capture reagent can selectively bind a SMDA. In one aspect, the SMDA capture reagent is an antibody, antigen, ligand, receptor, oligonucleotide, hapten, epitope, mimitope, and an aptamer. In one aspect, the SMDA capture reagent includes an antibody, or an epitope binding portion thereof that is capable of selectively binding a surface marker in or on the surface of the SMDA.

In one aspect, the target analyte capture reagent can selectively bind a target analyte of interest. In one aspect, the target analyte capture reagent is an antibody, antigen, ligand, receptor, oligonucleotide, hapten, epitope, mimitope, and an aptamer. In one aspect, the target analyte capture reagent is an antibody or epitope binding portion thereof that is capable of selectively binding to a target analyte released from the SMDA.

In one aspect, the support surface includes a plurality of capture reagents in a plurality of binding domains. In one aspect, the support surface includes a plurality of SMDA capture reagents in a plurality of SMDA binding domains. In one aspect, the support surface includes a plurality of target analyte capture reagents in a plurality of target analyte binding domains. In one aspect, the support surface includes a plurality of targeting reagent complements in a plurality of SMDA binding domains. In one aspect, the support surface includes a plurality of targeting reagent complements in a plurality of target analyte binding domains. In one aspect, the support surface includes a plurality of targeting reagent complements in a plurality of SMDA binding domains and a plurality of targeting reagent complements in a plurality of target analyte binding domains. In one aspect, a first support surface includes a plurality of targeting reagent complements in a plurality of SMDA binding domains. In one aspect, a second support surface includes a plurality of targeting reagent complements in a plurality of target analyte binding domains. In one aspect, a multiplexed format is used in which a plurality of SMDA or target analytes are isolated, detected, quantified or characterized using a plurality of capture reagents. In one aspect, one or more capture reagents are immobilized on a single component support surface in one or more binding domains.

In one aspect, one or more capture reagents are immobilized on a multi-component support surface, for example, particle or bead. In one aspect the multi-component support surface includes one or more coded particles or beads in which the coding identifies the capture reagent and SMDA or target analyte for a specific particle or bead. In one aspect, the method includes counting the number of beads that have a bound analyte.

In one aspect, the support surface includes one or more electrodes. In one aspect, one or more binding domains are formed on one or more electrodes of the support surface. In one aspect, the method includes an electrochemical or electrochemiluminescence assay.

In one aspect, one or more capture reagents are immobilized, directly or indirectly, on an electrode surface. In one aspect, one or more capture reagents are immobilized indirectly, for example, through the interaction between a targeting reagent attached to the capture reagent and a targeting reagent complement immobilized on the electrode. In one aspect, the electrode surface includes one or more discrete binding domains. In one aspect, the electrode is a component of a multi-well plate or a flow cell.

In one aspect, the electrode includes a conductive material, e.g., a metal such as gold, silver, platinum, nickel, steel, iridium, copper, aluminum, a conductive allow, or the like. In one aspect, the electrode includes oxide coated metals, e.g., aluminum oxide coated aluminum. In one aspect, the electrode includes working and counter electrodes which can be made of the same or different materials, e.g., a metal counter electrode and carbon working electrode. In one aspect, electrodes include carbon-based materials such as carbon, carbon black, graphitic carbon, carbon nanotubes, carbon fibrils, graphite, graphene, carbon fibers and mixtures thereof. In one aspect, the electrodes include elemental carbon, e.g., graphitic, carbon black, carbon nanotubes, etc. In one aspect, one or more electrodes may include conducting carbon-polymer composites, conducting particles dispersed in a matrix (e.g. carbon inks, carbon pastes, metal inks, graphene inks), or conducting polymers. In one aspect, the support surface includes an assay module, for example, a multi-well plate with one or m more electrodes (e.g., working or counter electrodes) that include carbon, e.g., carbon layers, or screen-printed layers of carbon inks.

### H. Detecting the Target Analyte

In one aspect, the target analyte is assayed. In one aspect, the assay is an ultrasensitive assay. In one aspect, the target analyte is assayed while immobilized on the support surface. In one aspect, the target analyte is assayed after the it is released from the support surface.

In one aspect, immobilized target analyte is subjected to a measuring step, which are known and include, for example, those disclosed in International Appl. No. PCT/US2015/030925, published as WO 2015/175856, which is incorporated by reference in its entirety. This application describes an ultrasensitive assay format for soluble proteins that marry a variation of proximity ligation amplification (PLA) with ECL detection to provide state-of-the-art sensitivity. The measuring step of the method can include imaging an optical signal from the surface to generate an image that includes a plurality of pixels, wherein each resolvable binding region maps to one or more pixels or groups of pixels in the image. Image analysis to identify pixels or sets of pixels having a signal indicative of a binding event (detection complex) can be accomplished using art recognized methods.

In one aspect, the method includes detecting, quantifying or characterizing a plurality of target agents. In one aspect, the method uses a support surface that includes a plurality of capture reagents distributed across a plurality of resolvable binding regions positioned on the support surface. A resolvable binding region may also be a microparticle within a plurality of microparticles. Under suitable conditions, a “resolvable binding region” is the minimal surface area associated with an individual binding event that can be resolved and differentiated from another area in which an additional individual binding event is occurring.

In one aspect, visualization or quantification of the target analyte is performed prior to releasing the target analyte from the support surface. In one aspect, visualization or quantification of the target analyte is performed after the target analyte is released from the support surface. In one aspect, the target analyte is detected using a detectable label, for example, an electrochemiluminescent or fluorescent label.

### I. Methods of Detection

The resolvable binding regions in which the SDMA or target analyte are immobilized can be optically interrogated, in whole or in part, i.e., each individual resolvable binding region can be individually optically interrogated or the entire surface including a plurality of resolvable binding regions can be imaged and one or more pixels or groupings of pixels within that image can be mapped to an individual resolvable binding region.

The resolvable binding regions exhibiting changes in their optical signature can be identified by a conventional optical detection system. Depending on the detected species (e.g., type of fluorescence entity, etc.) and the operative wavelengths, optical filters designed for a particular wavelength can be employed for optical interrogation of the resolvable binding regions. In one aspect where optical interrogation is used, the system can include more than one light source or a plurality of filters to adjust the wavelength or intensity of the light source. In one aspect, the optical signal from a plurality of resolvable binding regions is captured using a CCD camera. Other non-limiting examples of camera imaging systems that can be used to capture images include charge injection devices (CIDs), complementary metal oxide semiconductors (CMOSs) devices, scientific CMOS (sCMOS) devices, and time delay integration (TDI) devices, as will be known to those of ordinary skill in the art. In one aspect, a scanning mirror system coupled with a photodiode or photomultiplier tube (PMT) can be used for imaging.

Additional methods of interrogating a target analyte includes bioluminescence, and NMR. In one aspect, the target analyte is assessed by quantitative polymerase chain reaction, next generation sequencing, or both. Multiplexed measurement of analytes using electrochemiluminescence is described in U.S. Pat. Nos. 7,842,246 and 6,977,722, the disclosures of which are incorporated herein by reference in their entireties.

In one aspect, a method of conducting a multiplexed binding assay for a plurality of SDMA or target analytes is provided. Multiplex binding assays are known and include those described in U.S. Patent Publication No. 2016/0069872, filed Sep. 8, 2015, entitled METHODS FOR CONDUCTING MULTIPLEXED ASSAYS AND International Application No. WO 2020/227016, filed Apr. 30, 2020, entitled KTS FOR DETECTING ONE OR MORE TARGET NUCLEIC ACID ANALYTES IN A SAMPLE AND METHODS OF MAKING AND USING THE SAME, the disclosures of which are incorporated herein by reference in their entireties.

In a multiplex variant of the method described herein, the plate is coated with a plurality of different SMDA capture reagents, which may capture SMDA with different surface markers. In another multiplex variant method described above, the plate is coated with a plurality of different target analyte capture reagents, which may capture different target analytes of interest. The multiplexed methods described herein advantageously allow the same sample containing multiple SMDA or target analytes to be assayed in one experiment, which may help to reduce the amount of sample required and decrease sample-to-sample variability.

In one aspect, SMDA or target analyte is measured using an electrochemiluminescence-based assay, e.g. electrochemiluminescence (ECL) based immunoassays. The light generated by ECL labels can be used as a reporter signal in diagnostic procedures (Bard et al., U.S. Pat. No. 5,238,808, herein incorporated by reference). For example, an ECL label can be covalently coupled to a detection reagent such as an antibody, nucleic acid probe, receptor or ligand; the participation of the detection reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants).

Species that can be induced to emit ECL (ECL-active species) have been used as ECL labels, e.g., i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Species that participate with the ECL label in the ECL process are referred to herein as ECL coreactants. Commonly used coreactants include tertiary amines (e.g., see U.S. Pat. No. 5,846,485 and U.S. Provisional Application No. 62/787,892, filed on Jan. 3, 2019), oxalate, and persulfate for ECL from RuBpy and hydrogen peroxide for ECL from luminol (see, e.g., U.S. Pat. No. 5,240,863). In one aspect, the ECL coreactant is tripropylamine (TPA). In one aspect, the ECL coreactant is N-Butyldiethanolamine (BDEA). In one aspect, the ECL coreactant is N,N-dibutylethanolamine (DBAE). In one aspect, the ECL coreactant is included in a read buffer for the ECL assay. In one aspect, the read buffer includes an ECL coreactant and a surfactant. In one aspect, the surfactant is TRITON X-100. In one aspect, the read buffer does not include TRITON X-100. In one aspect, the surfactant does not disrupt a surface of the surface marker displaying agent. In one aspect, the surfactant does not disrupt a lipid bilayer membrane. In one aspect, the surfactant does not disrupt a membrane of an EV. In one aspect, the surfactant is BRIJ, TWEEN, PLURONIC or KOLLIPHOR. In one aspect, the surfactant is TWEEN. In one aspect, the read buffer does not include a surfactant.

In another aspect, the SMDA or target analyte is measured using a fluorescence-based assay. In one aspect, the fluorescent-based assay includes the use of fluorescent labels to detect the target analyte. In one aspect, the fluorescent label includes fluorescent species that can be used in single molecule fluorescence detection, e.g., fluorescence correlation spectroscopy, or fluorescence cross-correlation spectroscopy.

### IV. Kits

In one aspect, a kit is provided for isolating, detecting or quantifying one or more target analytes encapsulated in one or more surface marker displaying agents (SMDA) in a sample. In one aspect, the kit includes, in one or more vials, containers, or compartments:
a support surface with one or more binding domains;
a SMDA capture reagent that binds, directly or indirectly, a surface marker of the SMDA, wherein the SMDA capture reagent is or can be immobilized, directly or indirectly, on a SMDA binding domain; and
a target analyte capture reagent that binds, directly or indirectly, the target analyte, wherein the target analyte capture reagent is or can be immobilized, directly or indirectly, on a target analyte binding domain.

In one aspect, the support surface includes a plurality of binding domains. In one aspect, the support surface includes a single component with a plurality of binding domains. In one aspect, the support surface includes an array with a plurality of capture reagents immobilized in one or more binding domains. In one aspect, the support surface includes an array with a plurality of SMDA capture reagents immobilized in one or more SMDA binding domains. In one aspect, the support surface includes an array with a plurality of target analyte capture reagents immobilized in one or more target analyte binding domains. In one aspect, the support surface includes an array with a plurality of targeting reagent complements immobilized in one or more SMDA binding domains In one aspect, the support surface includes an array with a plurality of targeting reagent complements immobilized in one or more target analyte binding domains. In one aspect, the support surface includes a plate with multiple wells. In one aspect, each well includes at least one binding domain. In one aspect, each well includes (i) at least one SMDA binding domain that includes a SMDA capture reagent or a SMDA targeting reagent complement and (ii) at least one target analyte binding domain that includes a target analyte capture reagent or a target analyte targeting reagent complement. In one aspect, the support surface includes multiple components. In one aspect, the support surface includes a plurality of particles or beads. In one aspect each binding domain is on a separate particle or bead.

In one aspect, the kit includes a plurality of SMDA capture reagents that bind, directly or indirectly, a surface marker of a SMDA. In one aspect, the kit includes a plurality of target analyte capture reagents that bind, directly or indirectly, a target analyte. In one aspect, each binding domain on the support surface includes a different capture reagent. In one aspect, each SMDA binding domain on the support surface includes a different SMDA capture reagent. In one aspect, each target analyte binding domain on the support surface includes a different target analyte capture reagent. In one aspect, each SMDA binding domain on the support surface includes a different targeting reagent complement. In one aspect, each target analyte binding domain on the support surface includes a different targeting reagent complement.

In one aspect, the kit includes a SMDA capture reagent that selectively binds the surface marker of the SMDA. In one aspect, the SMDA capture reagent includes an antigen-binding substance, antigen, ligand, receptor, oligonucleotide, hapten, epitope, mimotope, or aptamer. In one aspect, the SMDA capture reagent includes an antigen-binding substance. In one aspect, the SMDA capture reagent is immobilized on the support surface. In one aspect, the SMDA capture reagent includes a targeting reagent that is capable of selectively binding to a targeting reagent complement immobilized in a SMDA binding domain on the support surface. In one aspect, the SMDA capture reagent includes a targeting reagent capable of binding to a corresponding targeting reagent complement immobilized on the SMDA binding domain. In one aspect, the targeting reagent and the targeting reagent complement include complementary oligonucleotides.

In one aspect, the SMDA capture reagent is an antibody, antigen, ligand, receptor, oligonucleotide, hapten, epitope, mimitope, or aptamer that selectively binds a SMDA and is immobilized on a SMDA binding domain of the support surface. In one aspect, the SMDA capture reagent is an antibody, antigen, ligand, receptor, oligonucleotide, hapten, epitope, mimitope, or aptamer that selectively binds a SMDA and is or can be immobilized on a SMDA binding domain of the support surface via the binding between a targeting reagent of the SMDA capture reagent and a targeting reagent complement immobilized on the support surface. In one aspect, the SMDA capture reagent is an antibody, or an epitope binding portion of an antibody.

In one aspect, the SMDA capture reagent includes a targeting reagent capable of binding to a corresponding targeting reagent complement immobilized in the SMDA binding domain. In one aspect, the targeting reagent and its corresponding targeting reagent complement include complementary oligonucleotides. In one aspect, the targeting reagent and targeting reagent complement are members of a binding pair, for example, avidin-biotin, streptavidin-biotin, antibody-hapten, antibody-antigen, antibody-epitope tag, nucleic acid-complementary nucleic acid, aptamer-aptamer target, and receptor-ligand. In one aspect, the targeting reagent is biotin and the targeting reagent complement is streptavidin.

In one aspect, the kit includes a target analyte capture reagent that selectively binds the target analyte. In one aspect, the target analyte capture reagent includes an antigen-binding substance, antigen, ligand, receptor, oligonucleotide, hapten, epitope, mimotope, or aptamer. In one aspect, the target analyte capture reagent includes an antigen-binding substance. In one aspect, the target analyte capture reagent is or can be immobilized on the support surface. In one aspect, the target analyte capture reagent includes a targeting reagent capable of binding to a corresponding targeting reagent complement immobilized on the target analyte binding domain. In one aspect, the targeting reagent and the targeting reagent complement include complementary oligonucleotides.

In one aspect, the kit includes a support surface that includes one or more SMDA binding domains on which one or more SMDA capture reagents or one or more targeting reagent complements are immobilized. In one aspect, the kit includes a support surface that includes one or more target analyte binding domains on which one or more target analyte capture reagents or one or more targeting reagent complements are immobilized. In one aspect, the kit includes a support surface that includes one or more SMDA binding domains on which one or more SMDA capture reagents or one or more targeting reagent complements are immobilized and one or more target analyte binding domains on which one or more target analyte capture reagents or one or more targeting reagent complements are immobilized. In one aspect, the kit includes a first support surface that includes one or more SMDA binding domains on which one or more SMDA capture reagents or targeting reagent complements are immobilized and a second support surface that includes one or more target analyte binding domains on which one or more target analyte capture reagents or targeting reagent complements are immobilized.

In one aspect, the target analyte capture reagent is an antibody, antigen, ligand, receptor, oligonucleotide, hapten, epitope, mimitope, or aptamer that selectively binds a target analyte and is or can be immobilized on a target analyte binding domain of the support surface. In one aspect, the target analyte capture reagent is an antibody, antigen, ligand, receptor, oligonucleotide, hapten, epitope, mimitope, or aptamer that selectively binds a target analyte and is or can be immobilized on a target analyte binding domain of the support surface via the binding between a targeting reagent of the target analyte capture reagent and a targeting reagent complement immobilized on the support surface. In one aspect, the target analyte capture reagent is an antibody, or an epitope binding portion of an antibody.

In one aspect, the target analyte capture reagent includes a targeting reagent capable of binding to a corresponding targeting reagent complement immobilized on the target analyte binding domain. In one aspect, the targeting reagent and the targeting reagent complement include complementary oligonucleotides. In one aspect, the targeting reagent and targeting reagent complement are members of a binding pair, for example, avidin-biotin, streptavidin-biotin, antibody-hapten, antibody-antigen, antibody-epitope tag, nucleic acid-complementary nucleic acid, aptamer-aptamer target, and receptor-ligand. In one aspect, the targeting reagent is biotin and the targeting reagent complement is streptavidin.

In one aspect, the surface of the kit includes a plurality of distinct binding domains and the SMDA capture reagent and target analyte capture reagent are located in two distinct binding domains on the surface. In one aspect, the surface of the kit includes a plurality of distinct binding domains that include a SMDA capture reagent and a target analyte capture reagent in the same binding domain. In one aspect, the surface includes a plurality of distinct binding domains and different targeting reagent complements are immobilized in different binding domains. In one aspect, the support surface includes one or more anchoring oligonucleotides immobilized in one or more binding domains. In one aspect, one or more anchoring oligonucleotides are immobilized in one or more SMDA binding domains. In one aspect, one or more anchoring oligonucleotides are immobilized in one or more target analyte binding domains.

In one aspect, the support surface in the kit includes an electrode. In one aspect, the support surface is a carbon-based electrode. In one aspect, the kit includes a multi-well plate assay consumable, and each well of the plate includes a carbon ink electrode.

In one aspect, the kit includes one or more buffers. In one aspect, the kit includes one or more of a wash buffer, an assay buffer, and a read buffer. In one aspect, the same buffer can be used for the wash, assay, and detection (i.e., “read”) steps. In one aspect, the kit includes a Tris buffer or a phosphate buffer. Non-limiting examples of wash buffers, assay buffers, or read buffers include phosphate buffer, Tris buffer, HEPES buffer, and the like. In one aspect, the wash buffer or the read buffer includes a surfactant. In one aspect, the surfactant includes TRITON-X. In one aspect, the surfactant includes TWEEN-20. In one aspect, the wash buffer or the read buffer includes a co-reactant. In one aspect, the co-reactant is tripropylamine (TPA). In one aspect, the read buffer is a Tris buffer including TRITON-X and TPA. In one aspect, the read buffer includes N-Butyldiethanolamine (BDEA). In one aspect, the read buffer includes N,N-dibutylethanolamine (DBAE). In one aspect, the read buffer includes a surfactant that does not disrupt a surface of the surface marker displaying agent. In one aspect, the read buffer includes a surfactant that does not disrupt a lipid bilayer membrane. In one aspect, the read buffer includes a surfactant that does not disrupt a membrane of a SMDA. In one aspect, the surfactant is BRIJ, TWEEN, PLURONIC or KOLLIPHOR. In one aspect, the read buffer does not include a surfactant. In one aspect, the read buffer is a read buffer provided in, e.g., U.S. Provisional Application No. 62/787,892, filed on Jan. 3, 2019.

In one aspect, the kit includes a SMDA detection reagent for detecting the SMDA. In one aspect, the SMDA detection reagent includes an antigen-binding substance, antigen, ligand, receptor, oligonucleotide, hapten, epitope, mimotope, or aptamer. In one aspect, the SMDA detection reagent includes an oligonucleotide sequence. In one aspect, the SMDA detection reagent includes a detectable label.

In one aspect, the kit includes a target analyte detection reagent for detecting the target analyte. In one aspect, the target analyte detection reagent includes an antigen-binding substance, antigen, ligand, receptor, oligonucleotide, hapten, epitope, mimotope, or aptamer. In one aspect, the target analyte detection reagent includes an oligonucleotide sequence. In one aspect, the target analyte detection reagent includes a detectable label.

In one aspect, the detectable label is an electrochemiluminescence (ECL) label. Examples of ECL labels include, but are not limited to, i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds.

In one aspect, the detectable label is a radioactive, fluorescent, chemiluminescent, electrochemiluminescent, light absorbing, light scattering, electrochemical, magnetic or enzymatic label.

In one aspect, the kit includes one or more ECL coreactants, including, for example, tertiary amines, oxalate, and persulfate for ECL from RuBpy and hydrogen peroxide for ECL from luminol. In one aspect, the ECL coreactant is tripropylamine (TPA). In one aspect, the ECL coreactant is N-Butyldiethanolamine (BDEA). In one aspect, the ECL coreactant is N,N-dibutylethanolamine (DBAE). In one aspect, the ECL coreactant is included in a read buffer for the ECL assay.

In one aspect, the kit includes one or more buffers, for example, a wash buffer, a hybridization buffer, a binding buffer, or a read buffer. In one aspect, the kit includes a read buffer. In one aspect, the read buffer includes an ECL coreactant.

### V. Incorporation by Reference

All references cited herein, including patents, patent applications, papers, text books and the like, and the references cited therein, to the extent that they are not already, are hereby incorporated herein by reference in their entirety for all purposes.

### VI. Examples

### Example 1: SMDA and Target Analyte Capture and Analysis

The following is an overview of a method for detecting a target analyte encapsulated in a SMDA using an ECL-based sandwich immunoassays.

SMDA are captured on a solid support using affinity ligands that target known protein or carbohydrate moieties on the SMDA surface, such as the tetraspanin proteins, which are hypothesized to be present on the surface of most EVs. Antibodies are used to capture SMDA from a fluid sample and are displayed on a plate surface. MSD GOLD™ Streptavidin plates can be used to display biotinylated antibodies. Alternately, plates with directly coated antibodies can be used. In one aspect, solid-phase immunoaffinity capture is performed using beads as the solid phase.

Capture antibodies with reactivity to one or more SMDA surface markers serve as SMDA capture reagent and are immobilized and displayed on the surface of MSD plate electrodes, using either direct coating, biotinylated antibodies on streptavidin-coated plates, or a multispot system as described in U.S. Pat. Nos. 10,201,812; 7,842,246 and 6,977,722 (the disclosure of which are hereby incorporated by reference in their entireties).

A fluid sample suspected to contain one or more SMDA encapsulating one or more target analytes of interest is applied to the surface of the plate wells. The wells of the plate may be prefilled with a small amount of a diluent to improve the assay characteristics, for example, DPBS with 2% bovine serum albumin cam be used as the assay diluent and added at a ratio of 1:1 with the sample.

The plate is incubated to allow SMDA to be captured by the immobilized antibodies having reactivity to one or more SMDA surface markers (a first capture reagent) and the wells are washed with first wash to remove of unwanted components of the sample from the plate wells. Suitable washes include water or a wash buffer, for example, a wash buffer containing a small amount of Tween-20 (0.05% v/v) to aid in removal of unwanted components from the surface of the plate wells. (FIG. 1A)

After the plate wells are washed, the captured SMDA are lysed with a lysis reagent that is compatible with the assay conditions. Suitable lysis reagents include, for example, a detergent such as TRITON X-100. (FIG. 1B) A target analyte capture reagent is added to the plate wells and the plate is incubated to allow target analyte to be captured by the target analyte capture reagent. (FIG. 1B) In one aspect, the target analyte capture reagent is a biotinylated capture antibody that binds to the target analyte and which also binds to the streptavidin coated plate well surface.

It is possible for unwanted components from the SMDA that are not the target analyte to non-selectively bind to the plate or electrode surface. These unwanted components from the SMDA are removed with a second wash. Suitable washes include water or a wash buffer.

Detection reagent is added in a diluent and incubated for a sufficient time to allow a significant fraction of the immobilized target analyte to be decorated and the target analyte is assayed using a standard assay or an ultrasensitive assay.

### Example 2: SARS-COV-2 Capture and Target Analyte Analysis

The following is an overview of a method for detecting SARS-COV-2 and a target analyte encapsulated in SARS-COV-2 using an ECL-based immunoassay.

A patient sample suspected of containing SARS-COV-2 is applied to an assay plate and the SARS-COV-2 virus is captured on the assay plate surface via an immobilized affinity ligand that specifically binds to a viral protein, for example, an antibody that specifically binds to a viral spike protein.

The plate is washed to remove unwanted components and a labeled SARS-COV-2 detection reagent that specifically binds to the spike protein is used to detect SARS-COV-2 captured and immobilized on the plate surface.

The captured SARS-COV-2 is then contacted with a lysis reagent under conditions in which target analyte encapsulated by the SARS-COV-2 is released. The released target analyte is then contacted with a target analyte capture reagent and immobilized on the plate surface. In one aspect, the target analyte encapsulated by SARS-COV-2 is an internal nucleocapsid protein. In one aspect, the target analyte encapsulated by SARS-COV-2 is a viral genomic sequence. In one aspect, the target analyte encapsulated by SARS-COV-2 is a viral DNA or RNA sequence. In one aspect, the target analyte encapsulated by SARS-COV-2 is a mutant viral DNA or RNA sequence.

The plate may be washed again to remove unwanted components, including unbound analyte or other components of the SARS-COV-2 virus.

Detection reagent is added in a diluent and incubated for a sufficient time to allow a significant fraction of the immobilized target analyte to be decorated and the target analyte is assayed using a standard assay or an ultrasensitive assay.

### Example 3: SMDA and Target Analyte Capture and Analysis Using Streptavidin Coated Plates

The following is an overview of a method for detecting a target analyte encapsulated in a SMDA using an ECL-based sandwich immunoassays with MSD GOLD Streptavidin 96 well plates (Meso Scale Diagnostics, Rockville, MD).

Biotinylated capture antibodies directed to one or more SMDA surface markers(s) are displayed on the plate surface. The concentration of antibody added is limited so that only approximately half of the biotin-binding sites on the streptavidin-coated plate surface are occupied by biotinylated antibodies.

A fluid sample suspected to contain one or more SMDA encapsulating one or more target analytes of interest is applied to the plate wells. The wells of the plate may be prefilled with a small amount of a diluent to improve the assay characteristics, for example, DPBS with 2% bovine serum albumin can be used as the assay diluent and added at a ratio of 1:1 with the sample.

The plate is incubated to allow SMDA to be captured by the antibodies displayed on the plate well surface and the plate is washed to remove unwanted components of the sample from the plate. Here the unwanted components include unbound SMDAs, and soluble components, which may include soluble forms of the target analyte. Suitable washes include water or a wash buffer, for example, a wash buffer containing a small amount of Tween-20 (0.05% v/v) to aid in removal of unwanted components from the support surface. (FIG. 1A)

A biotinylated capture antibody, with specificity for the target analyte is then added to the plate wells in sufficient concentration to occupy all the remaining streptavidin sites on the plate well surface. Unbound antibody is washed away.

The captured SMDA are lysed with a lysis reagent that is compatible with the assay conditions. Suitable lysis reagents include, for example, a detergent such as TRITON X-100. (FIG. 1B). Upon lysis of the SMDA, the SMDA-encapsulated target analyte is released from the SMDA and is free to bind the second biotinylated capture antibody immobilized on the plate well surface. The plate is incubated for sufficient time to capture the analyte, typically 1-4 hours.

The plate may be washed again to remove unwanted components, including unbound analyte or other components of the SMDA.

Detection reagent is added in a diluent and incubated for a sufficient time to allow a significant fraction of the immobilized target analyte to be decorated and the target analyte is assayed using a standard assay or an ultrasensitive assay.

### Example 4: A Second Method for SMDA and Target Analyte Capture and Analysis Using Streptavidin Coated Plates

The following is an overview of a method for detecting a target analyte encapsulated in a SMDA using an ECL-based sandwich immunoassays with MSD GOLD Streptavidin 96 well plates. Here instead of limiting the amount of SMDA capture antibody to reserve biotin-binding sites on the plate surface for the analyte capture antibody, an addition of soluble streptavidin is used to add additional biotin-binding capacity to the plate after the SMDA is captured and washed.

Biotinylated capture antibodies with affinity for a SMDA surface protein are immobilized and displayed on the plate. The concentration of antibody added is sufficient to occupy substantially all of the biotin binding sites on the plate. As is typical of biotinylated antibody preparations, most of the antibody molecules in the reagent include more than one biotin group per molecule. After being immobilized and displayed on the plate surface, the antibodies contain biotin groups that remain free, that is, they are unassociated with a streptavidin group on the surface.

A fluid sample suspected to contain one or more SMDA encapsulating one or more target analytes of interest is applied to the plate wells. The wells of the plate may be prefilled with a small amount of a diluent to improve the assay characteristics, for example, DPBS with 2% bovine serum albumin cam be used as the assay diluent and added at a ratio of 1:1 with the sample.

The plate is incubated to allow SMDA to be captured by the immobilized antibodies displayed on the plate surface and the plate wells are washed to remove of unwanted components of the sample from the plate surface. Here the unwanted components include unbound SMDAs, and soluble components, which may include soluble forms of the target analyte. Suitable washes include water or a wash buffer, for example, a wash buffer containing a small amount of Tween-20 (0.05% v/v) to aid in removal of unwanted components from the surface of the wells. (FIG. 1A)

Streptavidin, or a similar multivalent biotin-binding reagent is added to the plate wells. Because streptavidin has multiple biotin-binding sites, it effectively converts the available biotin sites on the immobilized, surface-displayed capture antibodies into biotin-binding sites which can be used to immobilize and display a second capture antibody.

A second biotinylated capture antibody, with specificity for the target analyte is then added to the plate wells in sufficient concentration to occupy all the available streptavidin sites on the plate well surface (including biotin sites on immobilized SMDA capture antibodies). Unbound antibody is washed away.

The captured SMDA are lysed with a lysis reagent that is compatible with the assay conditions. Suitable lysis reagents include, for example, a detergent such as TRITON X-100. (FIG. 1B). Upon lysis of the SMDA, the SMDA-encapsulated analyte is released from the SMDA and is free to bind the second biotinylated capture antibody immobilized on the plate well surface. The plate is incubated for sufficient time to capture the analyte, typically 1-4 hours.

The plate wells can be washed again to remove unwanted components, including unbound analyte or other components of the SMDA.

Detection reagent is added in a diluent and incubated for a sufficient time to allow a significant fraction of the immobilized target analyte to be decorated and the target analyte is assayed using a standard assay or an ultrasensitive assay.

### Example 5: A Method for SMDA and Target Analyte Capture and Analysis Using a Combination of Directly Coated Antibodies and Directly Coated Streptavidin

The following is an overview of a method for detecting a target analyte encapsulated in a SMDA using an ECL-based sandwich immunoassays using plates with immobilized SMDA capture antibody and streptavidin.

Biotinylated SMDA capture antibodies and streptavidin are immobilized and displayed in each well of a multi-well plate. These can be displayed by direct coating of a mixture of antibody and streptavidin on the same electrode or antibody and streptavidin can be coated on different spots of a multispot system as described in U.S. Pat. Nos. 10,201,812; 7,842,246 and 6,977,722 (the disclosure of which are hereby incorporated by reference in their entireties).

A fluid sample suspected to contain one or more SMDA encapsulating one or more target analytes of interest is applied to the plate wells. The wells of the plate may be prefilled with a small amount of a diluent to improve the assay characteristics, for example, DPBS with 2% bovine serum albumin cam be used as the assay diluent and added at a ratio of 1:1 with the sample.

The plate is incubated to allow SMDA to be captured by the antibodies displayed on the plate and the wells are washed to remove unwanted components of the sample from the plate. Here the unwanted components include unbound SMDAs, and soluble components, which may include soluble forms of the target analyte. Suitable washes include water or a wash buffer, for example, a wash buffer containing a small amount of Tween-20 (0.05% v/v) to aid in removal of unwanted components from the plate well surface. (FIG. 1A)

A biotinylated capture antibody, with specificity for the target analyte is then added to the plate wells in sufficient concentration to occupy all the streptavidin sites on the plate well surface. Unbound antibody is washed away.

The captured SMDA are lysed with a lysis reagent that is compatible with the assay conditions. Suitable lysis reagents include, for example, a detergent such as TRITON X-100. (FIG. 1B). Upon lysis of the SMDA, the SMDA-encapsulated analyte is released from the SMDA and is free to bind the biotinylated capture antibody immobilized on the plate well surface. The plate is incubated for sufficient time to capture the analyte, typically 1-4 hours.

The plate wells can be washed again to remove unwanted components, including unbound analyte or other components of the SMDA.

Detection reagent is added in a diluent and incubated for a sufficient time to allow a significant fraction of the immobilized target analyte to be decorated and the target analyte is assayed using a standard assay or an ultrasensitive assay.

### Example 6: A Method for SMDA and Target Analyte Capture and Analysis Using a MSD U-PLEX System

The following is an overview of a method for detecting a target analyte encapsulated in a SMDA using an ECL-based sandwich immunoassays with the MSD U-PLEX™ system to display antibodies on the plate well surface.

One or more biotinylated SMDA capture antibodies are complexed with U-PLEX linkers and immobilized and displayed on one or more spots (binding domains) in each well of the U-PLEX multi-well plate. At least one spot of the U-PLEX plate is left uncoated and reserved for later incorporation of an analyte capture antibody.

A fluid sample suspected to contain one or more SMDA encapsulating one or more target analytes of interest is applied to the plate wells. The wells of the plate may be prefilled with a small amount of a diluent to improve the assay characteristics, for example, DPBS with 2% bovine serum albumin cam be used as the assay diluent and added at a ratio of 1:1 with the sample.

The plate is incubated to allow SMDA to be captured by the immobilized SMDA capture antibodies displayed on the well surface and the wells are washed to remove unwanted components of the sample from the plate. Here the unwanted components include unbound SMDAs, and soluble components, which may include soluble forms of the target analyte. Suitable washes include water or a wash buffer, for example, a wash buffer containing a small amount of Tween-20 (0.05% v/v) to aid in removal of unwanted components from the plate well surface. (FIG. 1A)

One or more capture antibody with specificity for one or more target analytes is then complexed with U-PLEX linkers and added to the plate wells where it is immobilized and displayed on one or more of the reserved spots. The assay can be performed as a singleplex assay (i.e., to detect a single target analyte) or as a multiplex assay (i.e., to detect a plurality of target analytes). Unbound antibody is washed away.

The captured SMDA are lysed with a lysis reagent that is compatible with the assay conditions. Suitable lysis reagents include, for example, a detergent such as TRITON X-100. (FIG. 1B). Upon lysis of the SMDA, the SMDA-encapsulated analyte is released from the SMDA and is free to bind the analyte capture antibody immobilized on the plate well surface. The plate is incubated for sufficient time to capture the analyte, typically 1-4 hours.

The plate wells can be washed again to remove unwanted components, including unbound analyte or other components of the SMDA.

Detection reagent is added in a diluent and incubated for a sufficient time to allow a significant fraction of the immobilized target analyte to be decorated and the target analyte is assayed using a standard assay or an ultrasensitive assay.

### Example 7. Use of Bead-Based Assays for SMDA Cargo

The following is an overview of a method for detecting a target analyte encapsulated in a SMDA using a bead-based immunoassay.

SMDA capture antibodies are immobilized and displayed on one or more sets of beads, e.g. magnetic streptavidin-coated beads, or Luminex xMAP beads using methods known in the art.

A fluid sample suspected to contain one or more SMDA encapsulating one or more target analytes of interest is applied to the beads in a binding buffer. The binding buffer may contain components to improve the assay characteristics, for example, 2% bovine serum.

The beads are incubated to allow SMDA to be captured by the antibodies displayed on the bead surface and the beads are washed to remove unwanted components of the sample from the beads. Here the unwanted components include unbound SMDAs, and soluble components, which may include soluble forms of the target analyte. Suitable washes include water or a wash buffer, for example, a wash buffer containing a small amount of Tween-20 (0.05% v/v) to aid in removal of unwanted components from the surface of the beads. (FIG. 1A)

One or more additional sets of antibody-coated beads is added to the sample. These beads have antibodies with specificity towards the target analyte.

A lysis buffer that is compatible with the assay conditions is added to the bead-containing sample to lyse the captured SMDA. Suitable lysis reagents include, for example, a detergent such as TRITON X-100. (FIG. 1B). Upon lysis of the SMDA, the SMDA-encapsulated target analyte is released from the SMDA and is free to bind the target analyte capture antibody immobilized on the beads added in the second bead addition. The beads are incubated for sufficient time to capture the target analyte, typically 1-4 hours.

The beads can be washed again to remove unwanted components, including unbound analyte or other components of the SMDA.

Detection reagent is added in a diluent and incubated for a sufficient time to allow a significant fraction of the immobilized target analyte to be decorated and the target analyte is assayed using a standard assay or an ultrasensitive assay. This may be an ECL assay, fluorescent assay, or colorometric assay. The beads may be assessed in bulk using e.g. a plate reader or one at a time using e.g. a cytometer.

What is claimed is:

1. A method of isolating one or more target analytes of interest encapsulated by one or more surface marker displaying agents (SMDA) in a sample, comprising:
a) contacting a support surface with the sample comprising the SMDA encapsulating the target analyte, wherein the support surface comprises a plurality of binding domains and the SMDA selectively binds, either directly or indirectly to a SMDA binding domain on the support surface, wherein the SMDA is immobilized on the support surface;
b) removing unwanted components of the sample from the support surface;
c) contacting the support surface with a lysis reagent to lyse the SMDA and release the encapsulated target analyte; and
d) allowing the target analyte to selectively bind, either directly or indirectly, to a target reagent binding domain on the support surface, wherein the target analyte is immobilized on the support surface.

2. The method of claim 1, wherein the SMDA selectively binds to a SMDA capture reagent immobilized on the SMDA binding domain on the support surface.

3. The method of claim 1, wherein the SMDA selectively binds to a SMDA capture reagent that comprises a targeting reagent that is capable of selectively binding to a targeting reagent complement immobilized on the SMDA binding domain on the support surface.

4. The method of claim 1, wherein the target analyte selectively binds to a target analyte capture reagent immobilized on the target analyte binding domain on the support surface.

5. The method of claim 1, wherein the target analyte selectively binds to a capture reagent that comprises a targeting reagent that is capable of selectively binding to a targeting reagent complement immobilized on the target analyte binding domain.

6. The method of claim 2, wherein the SMDA capture reagent comprises a SMDA capture reagent that selectively binds to a surface marker of the SMDA.

7. The method of claim 6, wherein the SMDA capture reagent is immobilized on the SMDA binding domain of the support surface.

8. The method of any one of claims 5 to 7, wherein the SMDA capture reagent comprises an antigen-binding substance, antigen, ligand, receptor, oligonucleotide, hapten, epitope, mimotope, or aptamer.

9. The method of any one of claims 5 to 8, wherein the SMDA capture reagent comprises an antigen-binding substance for a surface marker on the SMDA.

10. The method of claim 9, wherein the SMDA capture reagent comprises an antigen-binding substance that selectively binds a disease-specific surface marker on the SMDA.

11. The method of any one of claims 5 to 10, wherein the SMDA capture reagent is releasably immobilized on the SMDA binding domain.

12. The method of any of claims 5 to 10, wherein the SMDA capture reagent comprises a first targeting reagent that selectively binds to a first targeting reagent complement immobilized on the first binding domain of the support surface.

13. The method of claim 12, wherein the first targeting reagent and the first targeting reagent complement comprise complementary oligonucleotide sequences.

14. The method of claim 5, wherein the SMDA capture reagent is releasably bound to the SMDA binding domain by a labile linker.

15. The method of claim 14, wherein the labile linker is a heat-labile linker, a photolabile linker, or a chemically labile linker.

16. The method of claim 14, wherein the labile linker comprises an oligonucleotide comprising a restriction site cleavable by a restriction endonuclease.

17. The method of claim 1, wherein the sample includes target analyte that is encapsulated in the SMDA and nonencapsulated target analyte.

18. The method of claim 17, wherein the nonencapsulated target analyte is an unwanted component in the sample.

19. The method of claim 18, wherein the method comprises contacting the sample with a blocking reagent to remove, destroy or block the unwanted nonencapsulated target analyte in the sample.

20. The method of claim 19, wherein the blocking reagent specifically binds to the nonencapsulated target analyte.

21. The method of claim 19, wherein the blocking reagent includes an enzyme that degrades the nonencapsulated target analyte.

22. The method of any one of claims 14 to 16, further comprising releasing the SMDA capture reagent from the support surface after the support surface is contacted with the lysis reagent.

23. The method of claim 1, comprising contacting the immobilized SMDA with a SMDA detection reagent to detect or quantify the SMDA.

24. The method of claim 23, comprising contacting the immobilized SMDA with the SMDA detection reagent before the support surface is contacted with the lysis reagent.

25. The method of claim 23, comprising contacting the immobilized SMDA with the SMDA detection reagent after the support surface is contacted with the lysis reagent.

26. The method of claim 1, wherein the support surface comprises a target analyte capture reagent immobilized on the target analyte binding domain.

27. The method of claim 1, comprising contacting the support surface with a target analyte capture reagent that selectively binds to the target analyte after the support surface is contacted with the SMDA and unwanted components of the sample are removed from the support surface.

28. The method of claim 1, comprising contacting the support surface with a target analyte capture reagent that selectively binds to the target analyte, wherein the target analyte capture reagent is or can be immobilized on the target analyte binding domain of the support surface.

29. The method of claim 28, wherein the target analyte capture reagent comprises an antigen-binding substance, antigen, ligand, receptor, oligonucleotide, hapten, epitope, mimotope, or aptamer.

30. The method of claim 28 or 29, wherein the target analyte capture reagent comprises an antigen-binding substance for the target analyte.

31. The method of any one of claims 28 to 30, wherein the target analyte capture reagent comprises a second targeting reagent, and the target analyte binding domain comprises a second targeting reagent complement that comprises a binding partner of the second targeting reagent.

32. The method of claim 31, wherein the second targeting reagent and the second targeting reagent complement comprise complementary oligonucleotide sequences.

33. The method of claim 31, wherein one or more binding domains on the support surface comprise different targeting reagent complements.

34. The method of claim 1, wherein the unwanted components of the sample do not bind to the support surface or the SMDA or target analyte capture reagents.

35. The method of claim 1, further comprising removing unwanted components of the SMDA from the support surface after the SMDA is lysed.

36. The method of claim 35, wherein the unwanted components of the SMDA do not bind to the support surface or the SMDA or target analyte capture reagents.

37. The method of any one of claims 1 to 36, further comprising conducting an assay to detect, quantitate, or both, one or more target analytes.

38. The method of any one of claims 1 to 37, wherein the SMDA encapsulates more than one target analyte of interest.

39. The method of claim 38, wherein the support surface comprises a different target analyte binding domain with a different target analyte capture reagent for each target analyte.

40. The method of claim 39, wherein each target analyte selectively binds, either directly or indirectly, their corresponding target analyte capture reagent.

41. The method of claim 40, comprising contacting the support surface with a plurality of different target analyte capture reagents, wherein each target analyte capture reagent selectively binds one of the target analytes of interest and is capable of selectively binding to a corresponding target analyte binding domain on the support surface.

42. The method of any of claims 39 to 41, further comprising assaying each target analyte.

43. The method of any one of claims 1 to 42, wherein the SMDA is a cell, a virus or viral particle, an organelle, a vesicle, or combination thereof.

44. The method of claim 43, wherein the SMDA comprises a cell.

45. The method of claim 44, wherein the cell comprises a bacterial cell.

46. The method of claim 44, wherein the cell comprises a eukaryotic cell.

47. The method of claim 44, wherein the eukaryotic cell comprises a human cell.

48. The method of claim 43, wherein the SMDA comprises a viral particle.

49. The method of claim 43, wherein the SMDA comprises a vesicle.

50. The method of claim 43, wherein the vesicle comprises an extracellular vesicle (EV).

51. The method of claim 50, wherein the EV is an exosome, a micro-vesicle, or a large-oncosome.

52. The method of any one of claims 1 to 51, wherein the target analyte is a protein, a nucleic acid, a carbohydrate, a lipid, a hormone, a metabolite, or a combination thereof.

53. The method of any one of claims 1 to 52, wherein the target analyte comprises a disease specific marker.

54. The method of claim 52 or 53, wherein the target analyte comprises a protein.

55. The method of claim 52 or 53, wherein the target analyte comprises a nucleic acid.

56. The method of claim 55, wherein the nucleic acid comprises DNA or RNA.

57. The method of claim 55 or 56, wherein the nucleic acid comprises miRNA.

58. The method of any one of claims 1 to 57, wherein the sample comprises purified SMDA.

59. The method of any one of claims 1 to 58, wherein the sample comprises a mammalian fluid, secretion, or excretion.

60. The method of claim 59, wherein the sample comprises a purified mammalian fluid, secretion, or excretion.

61. The method of claim 60, wherein the mammalian fluid, secretion, or excretion is purified by differential centrifugation, ultrafiltration, size-exclusion chromatography, immune-affinity, or combination thereof.

62. The method of any of claims 59 to 61, wherein the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions, and excretions.

63. The method of any one of claims 1 to 62, wherein assaying the target analyte comprises:
contacting the target analyte bound to the target analyte capture reagent with a target analyte detection reagent, wherein the target analyte detection agent comprises a nucleic acid probe, to form a target analyte complex comprising the target analyte capture reagent, the target analyte, and the target analyte detection reagent;
extending the nucleic acid probe of the analyte detection reagent to form an extended sequence, wherein the extended sequence comprises an oligonucleotide sequence that binds to the target analyte capture reagent immobilized on the target analyte binding domain of the support surface; and
measuring the amount of extended sequence immobilized on the support surface.

64. The method of claim 63, wherein the target analyte detection reagent comprises a detectable label.

65. The method of claim 63 or 64, wherein the target analyte capture reagent comprises an oligonucleotide sequence and the nucleotide probe comprises a nucleotide sequence that is complementary to oligonucleotide sequence of the target analyte capture reagent.

66. The method of any one of claims 63 to 65, wherein extending the nucleic acid probe comprises contacting the nucleic acid probe with a circular template oligonucleotide and forming the extended sequence by rolling circle amplification.

67. The method of any one of claims 63 to 66, wherein the target analyte detection reagent comprises a detectable label.

68. The method of any one of claims 63 to 66, wherein the extended sequence comprises a detection sequence complement that is complementary to a sequence of a detection oligonucleotide, and wherein measuring the amount of extended sequence immobilized on the support surface comprises contacting the extended sequence with a detection oligonucleotide and measuring the amount of detection oligonucleotide immobilized on the target analyte binding domain.

69. The method of claim 68, wherein the detection oligonucleotide comprises a detectable label.

70. The method of claim 69, wherein the detectable label comprises an electrochemiluminescent label (ECL).

71. The method of any one of claims 1 to 70, wherein the support surface comprises a single component comprising a plurality of binding domains.

72. The method of claim 71, wherein the support surface comprises a plate comprising multiple wells, and each well comprises at least one binding domain.

73. The method of claim 72, wherein the SMDA and target analyte binding domains are in the same well.

74. The method of claim 72, wherein the SMDA and target analyte binding domains are in different wells.

75. The method of any one of claims 1 to 74, wherein the support surface comprises multiple components, and each of the multiple components comprises at least one binding domain.

76. The method of claim 75, wherein the support surface comprises multiple beads, and each binding domain of the plurality of binding domains is on a separate bead.

77. The method of any one of claims 1 to 76, wherein the support surface comprises an electrode.

78. A kit for isolating, detecting, or quantifying one or more target analytes of interest encapsulated by one or more surface marker displaying agents (SMDA) in a sample, comprising, in one or more vials, containers, or compartments:
a) a support surface comprising a plurality of binding domains;
b) a SMDA capture reagent that binds, directly or indirectly, a surface marker of the SMDA, wherein the SMDA capture reagent is or can be immobilized on a SMDA binding domain; and
c) a target analyte capture reagent that binds, directly or indirectly, the target analyte, wherein the target analyte capture reagent is or can be immobilized on a target analyte binding domain.

79. The kit of claim 78, further comprising a SMDA capture reagent that selectively binds the surface marker of the SMDA.

80. The kit of claim 79, wherein the SMDA capture reagent comprises an antigen-binding substance, antigen, ligand, receptor, oligonucleotide, hapten, epitope, mimotope, or aptamer.

81. The kit of claim 80, wherein the SMDA capture reagent comprises an antigen-binding substance.

82. The kit of any of claims 78 to 81, further comprising a target analyte capture reagent that selectively binds the target analyte.

83. The kit of claim 82, wherein the target analyte capture reagent comprises an antigen-binding substance, antigen, ligand, receptor, oligonucleotide, hapten, epitope, mimotope, or aptamer.

84. The kit of claim 83, wherein the target analyte capture reagent comprises an antigen-binding substance

85. The kit of any one of claims 78 to 85, wherein one or more binding domains of the support surface comprise different capture reagents.

86. The kit of any one of claims 78 to 85, wherein the SMDA capture reagent is releasably bound to the support surface.

87. The kit of claim 78, wherein the SMDA capture reagent comprises a first targeting reagent capable of binding to a first targeting reagent complement on the SMDA binding domain, and the target analyte capture reagent comprises a second targeting reagent capable of binding to a second targeting reagent complement in the target analyte binding domain.

88. The kit of claim 87, wherein the targeting reagent and the targeting reagent complement comprise complementary oligonucleotides.

89. The kit of any one of claims 78 to 88, wherein the support surface comprises a single component comprising a plurality of binding domains.

90. The kit of claim 89, wherein the support surface comprises a plate comprising multiple wells, and each well comprises at least one binding domain.

91. The kit of any one of claims 78 to 88, wherein the support surface comprises a plurality of beads, and each binding domain of the plurality of binding domains is on a separate bead.

92. The kit of any one of claims 78 to 91, wherein the support surface comprises an electrode.
